(19) United States
(12) Patent Application Publication
(10) Pub. No.: US 2010/0093823 A1
(43) Pub. Date: Apr. 15, 2010

(54) STEROIDAL COMPOUNDS AS MELANOGENESIS MODIFIERS AND USES THEREOF

(76) Inventors: Seth J. Orlow, New York, NY (US); Li Ni Komatsu, Clarksville, MD (US)

Correspondence Address: WILSON, SONSINI, GOODRICH & ROSATI, 650 PAGE MILL ROAD, PALO ALTO, CA, 94304-1050, US

(21) Appl. No.: 12/433,322

(22) Filed: Apr. 30, 2009

Related U.S. Application Data
(60) Provisional application No. 61/049,114, filed on Apr. 30, 2008.

(51) Int. Cl. A61K 31/403; C12N 5/071; C07D 209/56; A61P 17/00

(52) U.S. Cl. 514/410; 435/325; 548/418

(57) ABSTRACT

A method for the identification of compounds) that control melanin synthesis (melanogenesis), and the preparation and use of such compounds, and compositions and formulations thereof to modify (e.g., inhibit) melanin production are disclosed. A representative compound of the invention is defined by formula I:

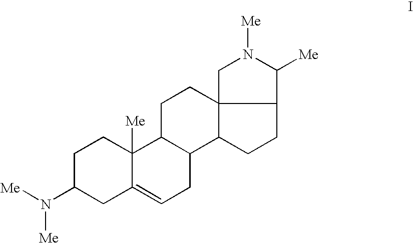

The compounds may be prepared as pharmaceutical and cosmetic compositions, and in one embodiment, may be prepared in combination with each other, and with other melanogenesis inhibiting formulations. The compounds, compositions and formulations of the invention may be used for the prevention and treatment of conditions that are causally related to aberrant melanogenesis activity including, by way of non-limiting example, hyperpigmentation and others.

### RELATED APPLICATIONS

The present application claims the benefit under 35 U.S.C. §119 of U.S. Provisional Application Ser. No. 61/049,114 filed Apr. 30, 2008. The contents of said provisional application is hereby incorporated by reference in its entirety.

### GOVERNMENT RIGHTS

This invention was made in part with government support under Grant No. AR41880 awarded by the National Institute of Health. Accordingly, the United States Government has certain rights in the invention.

### FIELD OF THE INVENTION

The present invention relates to the identification of compounds that modulate melanin synthesis (melanogenesis), and the use of such compounds and compositions thereof to modify (e.g., inhibit) melanin production. This invention also relates to methods for preventing and/or treating conditions that are causally related to aberrant melanogenesis activity, such as comprising (but not limited to) pigmentation abnormalities and hyperpigmentation, using the compounds of the invention. It is to be understood that such compounds may be used either alone or in combination with other compounds having the activity set forth herein.

### BACKGROUND OF THE INVENTION

Several publications and patent documents are referenced in this application in order to more fully describe the state of the art to which this invention pertains. The disclosure of each of these publications and documents is incorporated by reference herein.

Melanocytes synthesize melanin inside specialized organelles called melanosomes (reviewed in Orlow, 1998, in The Pigmentary System: Physiology and Pathophysiology 97, Oxford University Press, New York, Nordlund et al., eds). Melanosomes are formed by the fusion of two types of vesicles. Melanin is a dark biological pigment (biochrome) found in the skin, hair, feathers, scales, eyes, and some internal membranes of many animals that confers protection against ultraviolet radiation. Melanism refers to the deposition of melanin in the tissues of living animals, the chemistry of which depends on the metabolism of the amino acid tyrosine. More specifically, melanins are formed as an end product during metabolism of the amino acid tyrosine. Defects in the production of melanin and deposition of melanin (i.e., melanism) can result in pigmentation deficiencies such as albinism.

The ability to control melanin synthesis, which in turn, alters skin pigmentation may be used advantageously to address a variety of health-related conditions, as well as cosmetic objectives. Decreasing pigmentation is a desirable outcome in the treatment of disorders such as melasma, chloasma, post-inflammatory hyperpigmentation, solar lentigines, and the like.

The ability to modify skin coloring has generated considerable interest in many cultures. Inappropriate production or overproduction of melanin is considered a cosmetic problem by many individuals. In particular, the ability to remove hyperpigmentation, such as that found in age spots, freckles or aging skin generally, is of interest to individuals desiring a uniform complexion. Moreover, since chloasma, freckles, and pigmentary deposits that appear after over-exposure to the sun tend to occur or increase in frequency in middle aged and elderly individuals, such concerns are amplified in aging individuals. Indeed, with advancing years, these pigment deposits typically take longer to disappear and are more likely to become permanent. In certain areas of the world, general body whitening is also desirable.

A number of products have been developed to effect a decrease in skin pigmentation. One such product contains hydroquinone, a well known active substance for skin de-pigmentation, as described in U.S. Pat. No. 6,139,854. Hydroquinone can, however, have serious side effects if applied over a long period of time. The application of hydroquinone to skin may, for example, lead to permanent de-pigmentation, which results in increased photosensitivity of the skin upon exposure to ultraviolet light. Hydroquinone can be administered in combination with cortisone (which can thin the skin and cause other problems following facial administration), retinoic acid (an irritant), or glycolic acid (an irritant) to increase the efficacy of hydroquinone.

A variety of other substances have been proposed for use as regulators of skin pigmentation. Almost all of these substances work by either bleaching existing pigment or preventing new pigment synthesis by inhibiting the activity of tyrosinase, the principal rate limiting enzyme in the production of melanin. U.S. Pat. No. 6,123,959, for example, describes the use of aqueous compositions comprising liposomes and at least one competitive inhibitor of an enzyme involved in melanin synthesis. U.S. Pat. No. 5,132,740 describes the use of certain resorcinol derivatives as skin lightening agents. WO 99/64025 describes compositions for skin lightening which contain tyrosinase inhibiting extracts from dicotyledonous plant species indigenous to Canada. U.S. Pat. No. 5,580,549 describes an external preparation for skin lightening comprising 2-hydroxybenzoic acid derivatives and salts thereof as inhibitors of tyrosinase. WO 99/09011 describes an agent for inhibiting skin erythema and/or skin pigmentation, containing at least one carbostyril derivative and salts thereof. U.S. Pat. Nos. 5,214,028 and 5,389,611, describe lactoferrin hydrolyzates for use as tyrosinase inhibitory agents.

In WO 02 98347, Manga describes methods for identifying compounds that alter melanogenesis in melanogenic cells, more particularly, compounds that inhibit or enhance P protein function. This method is based, in part, on the observation that P protein function is required for proper cellular localization of tyrosinase and other melanosomal proteins, and is required for both full tyrosinase activity and melanogenesis in melanogenic cell types.

Orlow et al. describe screens for identifying compounds that inhibit or increase melanogenesis in melanogenic cells. See WO 01 1131. These studies were based upon the discovery that some compounds that inhibit melanogenesis do so by causing a mislocalization of tyrosinase, the key enzyme in melanin synthesis. Also, WO 2007/110415 is directed to the preparation of particular diacetyl trimers, and their use in compositions for cosmetic or therapeutic use, for decreasing melanin synthesis and concentration, for the lightening of skin.

Other studies are directed to methods and compositions for increasing melanogenesis. U.S. Pat. No. 5,352,440, for example, is directed to increasing melanin synthesis in melanocytes and increasing pigmentation by administration of certain diacylglycerol compounds. U.S. Pat. No. 5,532,001 is directed to increasing pigmentation in mammalian skin via administration of certain DNA fragments. U.S. Pat. No. 5,554,359 is directed to increasing levels of melanin in melanocytes by administration of lysosomotropic agents. U.S. Pat. Nos. 6,750,229 and 6,995,804 are directed to the identification of protease-activated receptor-2 (PAR-2) pathway and nitric oxide synthesis modulators, respectively, and their use in modulating pigmentation levels.

As described above, many methods have been proposed to achieve desired pigmentation levels of the skin. Such methods have included kojic acid, hydroquinone, retinoids and other chemical compounds for depigmentation purposes. The value of many of these compounds and compositions thereof, however, has been questionable. Precise application of all these compounds is necessary in order to achieve the desired result since a distinct line of demarcation between treated versus non-treated areas of the skin is frequently apparent. Moreover, many of these compounds cause skin irritation and, therefore, use of such compounds has undesirable side effects, particularly for long-term use.

As described herein, the present invention addresses the need for novel agents capable of regulating melanogenesis.

### SUMMARY OF THE INVENTION

In a first aspect, the present invention is directed to compounds that may be identified by cell-based assays, which compounds control melanogenesis. In brief, compounds were screened in cell-based assays to identify compounds capable of controlling, and particularly, inhibiting melanogenesis. Details pertaining to the screening assays are described in the Examples below. The results of the screening assays identified a plurality of compounds that modify (i.e. inhibit) melanogenesis, some of which were not previously known to exhibit such activity and others of which are known to affect melanogenesis. Notably, the confirmation of the activity of known modifiers of melanogenesis in the present screen corroborates the validity of the techniques and experimental approach.

The present invention is therefore directed to the identification of previously unidentified steroidal melanogenesis inhibitors, and their use in controlling (e.g. reducing) pigmentation in in vitro and in vivo applications. The novel melanogenesis modifiers of the present invention are represented by the formula I:

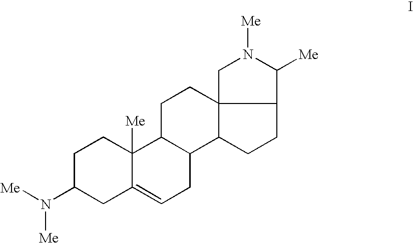

or a pharmaceutically acceptable salt, solvate or prodrug thereof, and stereoisomers, tautomers and isotopic variants thereof.

With respect to in vitro applications, test-tube based and additional cell-based assays may be used to test the ability of modified versions and/or steroidal compounds to alter melanogenesis. In vivo applications are directed to the administration of at least one of the novel steroidal melanogenesis modifier compounds to a subject desirous thereof or in need thereof to control, and particularly, to reduce pigmentation levels for prophylactic, therapeutic and/or cosmetic purposes.

In accordance with the present invention, therefore, a method is presented for effecting changes in mammalian skin pigmentation comprising topical application of at least one steroidal compound or a composition thereof to the skin of a mammal. The compositions of this invention may contain one or more of the steroidal compounds which have been identified as modifiers of melanogenesis.

More specifically and with respect to those compounds capable of reducing or inhibiting melanogenesis, the present invention encompasses a method for decreasing pigmentation in mammalian skin, hair or wool, and/or enhancing the brightening thereof, which comprises topically administering to the mammal an effective amount of one or more compounds described herein as a melanogenesis modifier.

Another aspect of this invention relates to the use of a compound of the invention in a therapeutic method, a pharmaceutical composition, and the manufacture of such composition, useful for the treatment of conditions involving aberrant or unwanted increases in production and consequent levels of melanin, and, in particular, diseases characteristic of abnormal melanogenesis, of which hyperpigmentation, acne, seborrheic keratoses, melasma and chloasma are particular such diseases or conditions. This invention also relates to processes for the preparation of the compounds of the invention.

In a particular embodiment, a melanogenesis modifier of the invention or a composition thereof may be applied to sites of hyperpigmentation including, without limitation, age spots, freckles, drug-induced hyperpigmentation, post-inflammatory hyperpigmentation as seen in acne, seborrheic keratoses, melasma and chloasma. For some individuals, body whitening over a larger area of the skin is desirable and may be achieved with a more generalized application of a melanogenesis inhibitor of the invention or a composition thereof.

In a further aspect, the present invention provides compositions, including cosmetic formulations, comprising a compound or compounds of the invention, and a suitable biocompatible or bioinert carrier, excipient or diluent. In this aspect of the invention, the cosmetic or pharmaceutical composition can comprise one or more of the compounds described herein. Moreover, the compounds of the present invention useful in the cosmetic and pharmaceutical compositions and treatment methods disclosed herein, are all pharmaceutically acceptable as prepared and used.

In a further aspect, the present invention provides compositions comprising a combination of a compound of the invention with various compounds or agents that may have a like effect on melanogenesis, such as, for example, other skin brightening agents, or skin lightening agents. In this aspect of the invention, the pharmaceutical composition can comprise one or more of the compounds described herein, individually or in combination with each other.

Other objects and advantages will become apparent to those skilled in the art from a consideration of the ensuing detailed description, which proceeds with reference to the following illustrative drawings.

### BRIEF DESCRIPTION OF THE DRAWINGS

FIG. 1A presents a series of photomicrographs taken of samples of MelanoDerm MEL-B, treated with a compound of the invention. All of the pictures were taken under identical conditions.

FIG. 1B is a bar graph presenting the results of quantitative analysis of the images presented in FIG. 1A. Analysis was performed using Adobe Photoshop CS2. The area of melanin granules was selected, and the melanin content was calculated as:

$$Melanin content = \frac{Black / Grey pixels \times mean density}{Total image pixels}$$

The final results were shown as percentage of control.

FIGS. 2A and 2B are photomicrographs depicting the resulting samples after Fontana-Masson staining. Quantitative analysis was performed using Adobe photoshop CS2 as indicated with respect to FIGS. 1A and 1B. The results of the staining of these samples indicates that melanin content of African-American skin equivalent decreases ˜50-60% following treatment with a compound of the invention. This result is graphically depicted in FIG. 2C, where C=control; L201=conesssine; Arb=Arbutin. In this figure, 1 image under each condition was shown for the day 10 treatment and 2 images for the day 13 treatment (the quantitative data for day 13 is the average of both images.

FIG. 3 is a graph depicting the overall results of the testing of a compound of the invention in the melanin assay with an African-American skin equivalent. The results show that melanin content of African-American skin equivalent decreases ˜30-40% with treatment of a compound of the invention. The data is the average of 2 tissues treated under each condition, and the melanin content of each tissue was determined individually.

FIG. 4 comprises two sets of photomicrographs (top views) taken of African-American skin equivalent (a different lot of MEL-B cells) at Days 10 and 13, treated with control and with the invention. The concentration of the inventive composition was 1.0 mM (equal to 356.6 μg/ml).

FIG. 5 comprises a series of photomicrographs depicting the results of Fontana-Masson staining of duplicate samples of African-American skin equivalent (the same lot as with FIG. 4), treated with control and the invention, at Days 10 and 13. The stained samples depict a demonstrable reduction in melanin content.

FIG. 6 is a graph presenting the results of the studies depicted in FIGS. 4 and 5, and demonstrating that melanin content of African-American skin equivalent decreases ˜30-40% with treatment by a compound of the invention.

FIG. 7 is a series of photomicrographs (top views) taken of Asian skin equivalent (MEL-A), at Days 13 and 16, treated with control and with the invention. The appearance of the samples demonstrates that treatment with the invention lightens the skin.

FIG. 8 is a series of photomicrographs depicting the results of Fontana-Masson staining of duplicate samples from a first study of Asian skin equivalent, at Days 13 and 16, treated with control and with the invention. As with the MEL-B samples, the stained MEL-A samples of the present study depict a demonstrable reduction in melanin content.

FIG. 9 is a series of photomicrographs depicting the results of Fontana-Masson staining of duplicate samples from a second study of Asian skin equivalent, at Days 13 and 16, treated with control and with the invention. As with samples from the first study, presented in FIG. 9, the stained MEL-A samples of the present study depict a demonstrable reduction in melanin content as well.

FIGS. 10A and 10B are graphs presenting the results of the studies depicted in FIGS. 8 and 9, and demonstrating that melanin content of Asian skin equivalent decreases 40% with treatment by a compound of the invention.

### DETAILED DESCRIPTION OF THE INVENTION

### Definitions

When describing the compounds, pharmaceutical compositions containing such compounds and methods of using such compounds and compositions, the following terms have the following meanings unless otherwise indicated. It should also be understood that any of the moieties defined forth below may be substituted with a variety of substituents, and that the respective definitions are intended to include such substituted moieties within their scope.

As used in this specification and the appended claims, the singular forms “a”, “an”, and “the” include plural references unless the context clearly dictates otherwise. Thus for example, reference to “the method” includes one or more methods, and/or steps of the type described herein and/or which will become apparent to those persons skilled in the art upon reading this disclosure.

“Acyl” refers to a group or radical —C(O)R²⁰, where R²⁰ is hydrogen, alkyl, cycloalkyl, cycloheteroalkyl, aryl, arylalkyl, heteroalkyl, heteroaryl, heteroarylalkyl as defined herein. Representative examples include, but are not limited to, formyl, acetyl, cyclohexylcarbonyl, cyclohexylmethylcarbonyl, benzyl, benzylcarbonyl and the like.

“Acylamino” refers to a group or radical —NR²¹C(O)R²², where R²¹ is hydrogen, alkyl, cycloalkyl, cycloheteroalkyl, aryl, arylalkyl, heteroalkyl, heteroaryl, heteroarylalkyl and R²² is hydrogen, alkyl, alkoxy, cycloalkyl, cycloheteroalkyl, aryl, arylalkyl, heteroalkyl, heteroaryl or heteroarylalkyl, as defined herein. Representative examples include, but are not limited to, formylamino, acetylamino, cyclohexylcarbonylamino, cyclohexylmethyl-carbonylamino, benzoylamino, benzylcarbonylamino and the like.

“Acyloxy” refers to the group or radical —OC(O)R²³ where R²³ is hydrogen, alkyl, aryl or cycloalkyl.

“Substituted alkenyl” includes those groups recited in the definition of “substituted” herein, and particularly refers to an alkenyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkoxy” refers to the group —OR²⁴ where R²⁴ is alkyl. Particular alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

“Substituted alkoxy” includes those groups recited in the definition of “substituted” herein, and particularly refers to an alkoxy group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, heteroaryl, hydroxyl, keto, nitro, thioalkoxy, substituted thioalkoxy, thioaryloxy, thioketo, thiol, alkyl-S(O)—, arylS(O)—, alkylS(O)₂— and aryl-S(O)₂—.

“Alkoxycarbonylamino” refers to the group —NR²⁵C(O)R²⁶ where R²⁵ is hydrogen, alkyl, aryl or cycloalkyl, and R²⁶ is alkyl or cycloalkyl.

“Alkyl” refers to monovalent saturated alkane radical groups particularly having up to about 11 carbon atoms, more particularly as a lower alkyl, from 1 to 8 carbon atoms and still more particularly, from 1 to 6 carbon atoms. The hydrocarbon chain may be either straight-chained or branched. This term is exemplified by groups such as methyl, ethyl, n-propyl, isopropyl, n-butyl, iso-butyl, tent-butyl, n-hexyl, n-octyl, tert-octyl and the like. The term “lower alkyl” refers to alkyl groups having 1 to 6 carbon atoms. The term “alkyl” also includes “cycloalkyls” as defined below.

“Substituted alkyl” includes those groups recited in the definition of “substituted” herein, and particularly refers to an alkyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, heteroaryl, keto, nitro, thioalkoxy, substituted thioalkoxy, thioaryloxy, thioketo, thiol, alkyl-S(O)—, aryl-S(O)—, alkyl-S(O)₂—, and aryl-S(O)₂—.

“Alkylene” refers to divalent saturated alkene radical groups having 1 to 11 carbon atoms and more particularly 1 to 6 carbon atoms which can be straight-chained or branched. This term is exemplified by groups such as methylene (—CH₂—), ethylene (—CH₂CH₂—), the propylene isomers (e.g., —CH₂CH₂CH₂— and —CH(CH₃)CH₂—) and the like.

“Substituted alkylene” includes those groups recited in the definition of “substituted” herein, and particularly refers to an alkylene group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, halogen, hydroxyl, keto, nitro, thioalkoxy, substituted thioalkoxy, thioaryloxy, thioketo, thiol, alkyl-S(O)—, aryl-S(O)—, alkyl-S(O)₂- and aryl-S(O)₂—.

“Alkenyl” refers to monovalent olefinically unsaturated hydrocarbyl groups preferably having 2 to 11 carbon atoms, particularly, from 2 to 8 carbon atoms, and more particularly, from 2 to 6 carbon atoms, which can be straight-chained or branched and having at least 1 and particularly from 1 to 2 sites of olefinic unsaturation. Particular alkenyl groups include ethenyl (—CH═CH₂), n-propenyl (—CH₂CH═CH₂), isopropenyl (—C(CH₃)═CH₂), vinyl and substituted vinyl, and the like.

“Alkenylene” refers to divalent olefinically unsaturated hydrocarbyl groups particularly having up to about 11 carbon atoms and more particularly 2 to 6 carbon atoms which can be straight-chained or branched and having at least 1 and particularly from 1 to 2 sites of olefinic unsaturation. This term is exemplified by groups such as ethenylene (—CH═CH—), the propenylene isomers (e.g., —CH═CHCH₂— and —C(CH₃)═CH— and —CH═C(CH₃)—) and the like.

“Alkynyl” refers to acetylenically or alkynically unsaturated hydrocarbyl groups particularly having 2 to 11 carbon atoms and more particularly 2 to 6 carbon atoms which can be straight-chained or branched and having at least 1 and particularly from 1 to 2 sites of alkynyl unsaturation. Particular non-limiting examples of alkynyl groups include acetylenic, ethynyl (—C≡CH), propargyl (—CH₂C≡CH), and the like.

“Substituted alkynyl” includes those groups recited in the definition of “substituted” herein, and particularly refers to an alkynyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Alkanoyl” or “acyl” as used herein refers to the group R²⁷—C(O)—, where R²⁷ is hydrogen or alkyl as defined above.

“Aryl” refers to a monovalent aromatic hydrocarbon group derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. Particularly, an aryl group comprises from 6 to 14 carbon atoms.

“Substituted Aryl” includes those groups recited in the definition of “substituted” herein, and particularly refers to an aryl group that may optionally be substituted with 1 or more substituents, for instance from 1 to 5 substituents, particularly 1 to 3 substituents, selected from the group consisting of acyl, acylamino, acyloxy, alkenyl, substituted alkenyl, alkoxy, substituted alkoxy, alkoxycarbonyl, alkyl, substituted alkyl, alkynyl, substituted alkynyl, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, nitro, thioalkoxy, substituted thioalkoxy, thioaryloxy, thiol, alkyl-S(O)—, aryl-S(O)—, alkyl-S(O)₂— and aryl-S(O)₂—.

“Fused Aryl” refers to an aryl having two of its ring carbon in common with a second aryl ring or with an aliphatic ring.

“Alkaryl” refers to an aryl group, as defined above, substituted with one or more alkyl groups, as defined above.

“Aralkyl” or “arylalkyl” refers to an alkyl group, as defined above, substituted with one or more aryl groups, as defined above.

“Aryloxy” refers to —O-aryl groups wherein “aryl” is as defined above.

“Alkylamino” refers to the group alkyl-NR²⁸R²⁹, wherein each of R²⁸ and R²⁹ are independently selected from hydrogen and alkyl.

“Arylamino” refers to the group aryl-NR³⁰R³¹, wherein each of R³⁰ and R³¹ are independently selected from hydrogen, aryl and heteroaryl.

“Alkoxyamino” refers to a radical —N(H)OR³² where R³² represents an alkyl or cycloalkyl group as defined herein.

“Alkoxycarbonyl” refers to a radical —C(O)-alkoxy where alkoxy is as defined herein.

“Alkylarylamino” refers to a radical NR³³R³⁴ where R³³ represents an alkyl or cycloalkyl group and R³⁴ is an aryl as defined herein.

“Alkylsulfonyl” refers to a radical —S(O)₂R³⁵ where R³⁵ is an alkyl or cycloalkyl group as defined herein. Representative examples include, but are not limited to, methylsulfonyl, ethylsulfonyl, propylsulfonyl, butylsulfonyl and the like.

“Alkylsulfinyl” refers to a radical —S(O)R³⁵ where R³⁵ is an alkyl or cycloalkyl group as defined herein. Representative examples include, but are not limited to, methylsulfinyl, ethylsulfinyl, propylsulfinyl, butylsulfinyl and the like.

“Alkylthio” refers to a radical —SR³⁵ where R³⁵ is an alkyl or cycloalkyl group as defined herein that may be optionally substituted as defined herein. Representative examples include, but are not limited to, methylthio, ethylthio, propylthio, butylthio, and the like.

“Amino” refers to the radical —NH₂.

“Substituted amino” includes those groups recited in the definition of “substituted” herein, and particularly refers to the group —N(R³⁶)₂ where each R³⁶ is independently selected from the group consisting of hydrogen, alkyl, substituted alkyl, alkenyl, substituted alkenyl, alkynyl, substituted alkynyl, aryl, cycloalkyl, substituted cycloalkyl, and where both R groups are joined to form an alkylene group. When both R groups are hydrogen, —N(R³⁶)₂ is an amino group.

“Aminocarbonyl” refers to the group —C(O)NR³⁷R³⁷ where each R³⁷ is independently hydrogen, alkyl, aryl and cycloalkyl, or where the R³⁷ groups are joined to form an alkylene group.

“Aminocarbonylamino” refers to the group —NR³⁸C(O)NR³⁸R³⁸ where each R³⁸ is independently hydrogen, alkyl, aryl or cycloalkyl, or where two R groups are joined to form an alkylene group.

“Aminocarbonyloxy” refers to the group —OC(O)NR³⁹R³⁹ where each R³⁹ is independently hydrogen, alkyl, aryl or cycloalkyl, or where the R groups are joined to form an alkylene group.

“Arylalkyloxy” refers to an —O-arylalkyl radical where arylalkyl is as defined herein.

“Arylamino” means a radical —NHR⁴⁰ where R⁴⁰ represents an aryl group as defined herein.

“Aryloxycarbonyl” refers to a radical —C(O)—O-aryl where aryl is as defined herein.

“Arylsulfonyl” refers to a radical —S(O)₂R⁴¹ where R⁴¹ is an aryl or heteroaryl group as defined herein.

“Azido” refers to the radical —N₃.

“Bicycloaryl” refers to a monovalent aromatic hydrocarbon group derived by the removal of one hydrogen atom from a single carbon atom of a parent bicycloaromatic ring system. Typical bicycloaryl groups include, but are not limited to, groups derived from indane, indene, naphthalene, tetrahydronaphthalene, and the like. Particularly, an aryl group comprises from 8 to 11 carbon atoms.

“Bicycloheteroaryl” refers to a monovalent bicycloheteroaromatic group derived by the removal of one hydrogen atom from a single atom of a parent bicycloheteroaromatic ring system. Typical bicycloheteroaryl groups include, but are not limited to, groups derived from benzofuran, benzimidazole, benzindazole, benzdioxane, chromene, chromane, cinnoline, phthalazine, indole, indoline, indolizine, isobenzofuran, isochromene, isoindole, isoindoline, isoquinoline, benzothiazole, benzoxazole, naphthyridine, benzoxadiazole, pteridine, purine, benzopyran, benzpyrazine, pyridopyrimidine, quinazoline, quinoline, quinolizine, quinoxaline, benzomorphan, tetrahydroisoquinoline, tetrahydroquinoline, and the like. Preferably, the bicycloheteroaryl group is between 9-11 membered bicycloheteroaryl, with 5-10 membered heteroaryl being particularly preferred. Particular bicycloheteroaryl groups are those derived from benzothiophene, benzofuran, benzothiazole, indole, quinoline, isoquinoline, benzimidazole, benzoxazole and benzdioxane.

“Carbamoyl” refers to the radical —C(O)N(R⁴²)₂ where each R⁴² group is independently hydrogen, alkyl, cycloalkyl or aryl, as defined herein, which may be optionally substituted as defined herein.

“Carboxy” refers to the radical —C(O)OH.

“Carboxyamino” refers to the radical —N(H)C(O)OH.

“Cycloalkyl” refers to cyclic hydrocarbyl groups having from 3 to about 10 carbon atoms and having a single cyclic ring or multiple condensed rings, including fused and bridged ring systems, which optionally can be substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, and multiple ring structures such as adamantanyl, and the like.

“Substituted cycloalkyl” includes those groups recited in the definition of “substituted” herein, and particularly refers to a cycloalkyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Cycloalkoxy” refers to the group —OR⁴³ where R⁴³ is cycloalkyl. Such cycloalkoxy groups include, by way of example, cyclopentoxy, cyclohexoxy and the like.

“Cycloalkenyl” refers to cyclic hydrocarbyl groups having from 3 to 10 carbon atoms and having a single cyclic ring or multiple condensed rings, including fused and bridged ring systems and having at least one and particularly from 1 to 2 sites of olefinic unsaturation. Such cycloalkenyl groups include, by way of example, single ring structures such as cyclohexenyl, cyclopentenyl, cyclopropenyl, and the like.

“Substituted cycloalkenyl” includes those groups recited in the definition of “substituted” herein, and particularly refers to a cycloalkenyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Fused Cycloalkenyl” refers to a cycloalkenyl having two of its ring carbon atoms in common with a second aliphatic or aromatic ring and having its olefinic unsaturation located to impart aromaticity to the cycloalkenyl ring.

“Cyanato” refers to the radical —OCN.

“Cyano” refers to the radical —CN.

“Dialkylamino” means a radical —NR⁴⁴R⁴⁵ where R⁴⁴ and R⁴⁵ independently represent an alkyl, substituted alkyl, aryl, substituted aryl, cycloalkyl, substituted cycloalkyl, cycloheteroalkyl, substituted cycloheteroalkyl, heteroaryl, or substituted heteroaryl group as defined herein.

“Ethenyl” refers to substituted or unsubstituted —(C═C)—.

“Ethylene” refers to substituted or unsubstituted —(C—C)—.

“Ethynyl” refers to —(C≡C)—.

“Halo” or “halogen” refers to fluoro, chloro, bromo and iodo. Preferred halo groups are either fluoro or chloro.

“Hydroxy” refers to the radical —OH.

“Nitro” refers to the radical —NO₂.

“Substituted” refers to a group in which one or more hydrogen atoms are each independently replaced with the same or different substituent(s). Typical substituents include, but are not limited to —X, —R⁴⁶—O⁻, ═O, —OR⁴⁶, —SR⁴⁶, —S⁻, ═S, —NR⁴⁶R⁴⁷, ═NR⁴⁶, —CX₃, —CF₃, —CN, —OCN, —SCN, —NO, —NO₂, ═N₂, —N₃, —S(O)₂O⁻, —S(O)₂OH, —S(O)₂R⁴⁶, —OS(O₂)O⁻, —OS(O)₂R⁴⁶, —P(O)(O⁻)₂, —P(O)(OR⁴⁶)(O), —OP(O)(OR⁴⁶)(OR⁴⁷), —C(O)R⁴⁶, —C(S)R⁴⁶, —C(O)OR⁴⁶, —C(O)NR⁴⁶R⁴⁷, —C(O)O⁻, —C(S)OR⁴⁶, —NR⁴⁸C(O)NR⁴⁶R⁴⁷, —NR⁴⁹C(NR⁴⁸)NR⁴⁶R⁴⁷ and —C(NR⁴⁸)NR⁴⁶R⁴⁷, where each X is independently a halogen; each R⁴⁶, R⁴⁷, R⁴⁸ and R⁴⁹ are independently hydrogen, alkyl, substituted alkyl, aryl, substituted alkyl, arylalkyl, substituted alkyl, cycloalkyl, substituted alkyl, cycloheteroalkyl, substituted cycloheteroalkyl, heteroalkyl, substituted heteroalkyl, heteroaryl, substituted heteroaryl, heteroarylalkyl, substituted heteroarylalkyl, —NR⁵⁰R⁵¹, —C(O)R⁵⁰ or —S(O)₂R⁵⁰ or optionally R⁵⁰ and R⁵¹ together with the atom to which they are both attached form a cycloheteroalkyl or substituted cycloheteroalkyl ring; and R⁵⁰ and R⁵¹ are independently hydrogen, alkyl, substituted alkyl, aryl, substituted alkyl, arylalkyl, substituted alkyl, cycloalkyl, substituted alkyl, cycloheteroalkyl, substituted cycloheteroalkyl, heteroalkyl, substituted heteroalkyl, heteroaryl, substituted heteroaryl, heteroarylalkyl or substituted heteroarylalkyl.

Examples of representative substituted aryls include the following

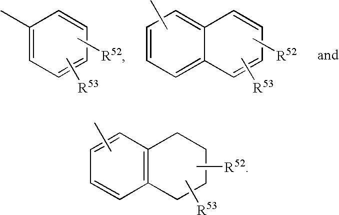

In these formulae one of R⁵² and R⁵³ may be hydrogen and at least one of R⁵² and R⁵³ is each independently selected from alkyl, alkenyl, alkynyl, cycloheteroalkyl, alkanoyl, alkoxy, aryloxy, heteroaryloxy, alkylamino, arylamino, heteroarylamino, NR⁵⁴COR⁵⁵, NR⁵⁴SOR⁵⁵, NR⁵⁴SO₂R⁵⁷, COOalkyl, COOaryl, CONR⁵⁴R⁵⁵, CONR⁵⁴OR⁵⁵, NR⁵⁴R⁵⁵, SO₂NR⁵⁴R⁵⁵, S-alkyl, S-alkyl, SOalkyl, SO₂alkyl, Saryl, SOaryl, SO₂aryl; or R⁵² and R⁵³ may be joined to form a cyclic ring (saturated or unsaturated) from 5 to 8 atoms, optionally containing one or more heteroatoms selected from the group N, O or S. R⁵⁴, R⁵⁵, and R⁵⁶ are independently hydrogen, alkyl, alkenyl, alkynyl, perfluoroalkyl, cycloalkyl, cycloheteroalkyl, aryl, substituted aryl, heteroaryl, substituted or hetero alkyl or the like.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g. heteroalkyl, cycloalkyl, e.g. cycloheteroalkyl, aryl, e.g. heteroaryl, cycloalkenyl, cycloheteroalkenyl, and the like having from 1 to 5, and especially from 1 to 3 heteroatoms.

“Heteroaryl” refers to a monovalent heteroaromatic group derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferably, the heteroaryl group is between 5-15 membered heteroaryl, with 5-10 membered heteroaryl being particularly preferred. Particular heteroaryl groups are those derived from thiophene, pyrrole, benzothiophene, benzofuran, indole, pyridine, quinoline, imidazole, oxazole and pyrazine.

Examples of representative heteroaryls include the following:

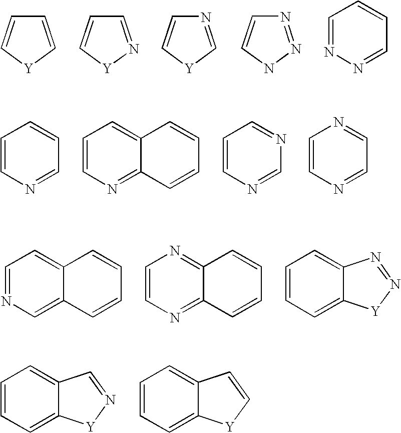

wherein each Y is selected from carbonyl, N, NR⁵⁸, O, and S; and R⁵⁸ is independently hydrogen, alkyl, cycloalkyl, cycloheteroalkyl, aryl, heteroaryl, heteroalkyl or the like.

As used herein, the term “cycloheteroalkyl” refers to a stable heterocyclic non-aromatic ring and fused rings containing one or more heteroatoms independently selected from N, O and S. A fused heterocyclic ring system may include carbocyclic rings and need only include one heterocyclic ring. Examples of heterocyclic rings include, but are not limited to, piperazinyl, homopiperazinyl, piperidinyl and morpholinyl, and are shown in the following illustrative examples:

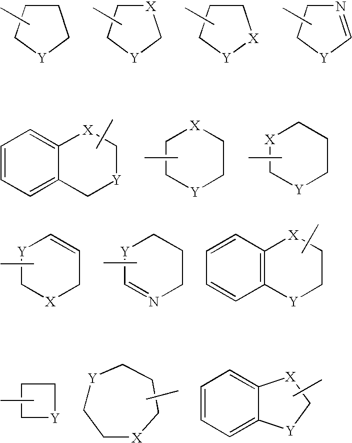

wherein each X is selected from CR⁵⁸, CR⁵⁸₂, NR⁵⁸, O and S; and each Y is selected from NR⁵⁸, O and S; and R⁵⁸ is independently hydrogen, alkyl, cycloalkyl, cycloheteroalkyl, aryl, heteroaryl, heteroalkyl or the like. These cycloheteroalkyl rings may be optionally substituted with one or more groups selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—. Substituting groups include carbonyl or thiocarbonyl which provide, for example, lactam and urea derivatives.

Examples of representative cycloheteroalkenyls include the following:

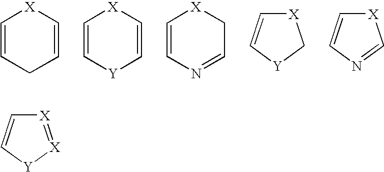

wherein each X is selected from CR⁵⁸, CR⁵⁸₂, NR⁵⁸, O and S; and each Y is selected from carbonyl, N, NR⁵⁸, O and S; and R⁵⁸ is independently hydrogen, alkyl, cycloalkyl, cycloheteroalkyl, aryl, heteroaryl, heteroalkyl or the like.

Examples of representative aryl having hetero atoms containing substitution include the following:

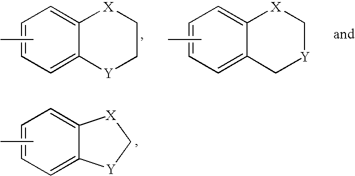

wherein each X is selected from CR⁵⁸₂, NR⁵⁸, O and S; and each Y is selected from carbonyl, NR⁵⁸, O and S; and R⁵⁸ is independently hydrogen, alkyl, cycloalkyl, cycloheteroalkyl, aryl, heteroaryl, heteroalkyl or the like.

“Hetero substituent” refers to a halo, O, S or N atom-containing functionality that may be present as an R⁴ in a R⁴C group present as substituents directly on the ring or rings of the compounds of this invention, or that may be present as a substituent in any “substituted” aryl and aliphatic groups present in the compounds.

Examples of hetero substituents include:

-halo,

—NO₂, —NH₂, —NHR⁵⁹, —N(R⁵⁹)₂,

—NRCOR, —NR⁵⁹SOR⁵⁹, —NR⁵⁹SO₂R⁵⁹, OH, CN,

—CO₂H,

—R⁵⁹—OH, —O—R⁵⁹, —COOR⁵⁹,

—CON(R⁵⁹)₂, —CONROR⁵⁹,

—SO₃H, —R⁵⁹—S, —SO₂N(R⁵⁹)₂,

—S(O)R⁵⁹, —S(O)₂R⁵⁹

wherein each R⁵⁹ is independently an aryl or aliphatic, optionally with substitution. Among hetero substituents containing R⁵⁹ groups, preference is given to those materials having aryl and alkyl R⁵⁹ groups as defined herein. Preferred hetero substituents are those listed above.

“Dihydroxyphosphoryl” refers to the radical —PO(OH)₂.

“Substituted dihydroxyphosphoryl” includes those groups recited in the definition of “substituted” herein, and particularly refers to a dihydroxyphosphoryl radical wherein one or both of the hydroxyl groups are substituted. Suitable substituents are described in detail below.

“Aminohydroxyphosphoryl” refers to the radical —PO(OH)NH₂.

“Substituted aminohydroxyphosphoryl” includes those groups recited in the definition of “substituted” herein, and particularly refers to an aminohydroxyphosphoryl wherein the amino group is substituted with one or two substituents. Suitable substituents are described in detail below. In certain embodiments, the hydroxyl group can also be substituted.

“Thioalkoxy” refers to the group —SR⁶⁰ where R⁶⁰ is alkyl.

“Substituted thioalkoxy” includes those groups recited in the definition of “substituted” herein, and particularly refers to a thioalkoxy group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, alkoxycarbonylamino, amino, substituted amino, aminocarbonyl, aminocarbonylamino, aminocarbonyloxy, aryl, aryloxy, azido, carboxyl, cyano, cycloalkyl, substituted cycloalkyl, halogen, hydroxyl, keto, nitro, thioalkoxy, substituted thioalkoxy, thioaryloxy, thioketo, thiol, alkyl-S(O)—, aryl-S(O)—, alkyl-S(O)₂— and aryl-S(O)₂—.

“Sulfanyl” refers to the radical HS—. “Substituted sulfanyl” refers to a radical such as RS— wherein R is any substituent described herein.

“Sulfonyl” refers to the divalent radical —S(O₂)—. “Substituted sulfonyl” refers to a radical such as R⁶¹—(O₂)S— wherein R⁶¹ is any substituent described herein. “Aminosulfonyl” or “Sulfonamide” refers to the radical H₂N(O₂)S—, and “substituted aminosulfonyl” “substituted sulfonamide” refers to a radical such as R⁶²₂N(O₂)S— wherein each R⁶² is independently any substituent described herein.

“Sulfone” refers to the group —SO₂R⁶³. In particular embodiments, R⁶³ is selected from H, lower alkyl, alkyl, aryl and heteroaryl.

“Thioaryloxy” refers to the group —SR⁶⁴ where R⁶⁴ is aryl.

“Thioketo” refers to the group ═S.

“Thiol” refers to the group —SH.

One having ordinary skill in the art of organic synthesis will recognize that the maximum number of heteroatoms in a stable, chemically feasible heterocyclic ring, whether it is aromatic or non aromatic, is determined by the size of the ring, the degree of unsaturation and the valence of the heteroatoms. In general, a heterocyclic ring may have one to four heteroatoms so long as the heteroaromatic ring is chemically feasible and stable.

As used herein, “mammal” refers to any member of the higher vertebrate animals comprising the class Mammalia, which includes, but is not limited to, humans.

As used herein, the term “melanogenesis inhibitor” is used to describe a compound or an agent, extract of a natural material, or the like, that is known or is identified herein as possessing the ability to inhibit melanogenesis in a melanocyte.

As used herein, an “amount effective” shall mean an amount sufficient to cover the region of skin, hair, fur, or wool surface where a change in pigmentation is desired.

“Pharmaceutically acceptable” means approved by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound of the invention that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non toxic organic or inorganic acids, such as hydrochloride, hydrobromide, tartrate, mesylate, acetate, maleate, oxalate and the like.

The term “pharmaceutically acceptable cation” refers to a non toxic, acceptable cationic counter-ion of an acidic functional group. Such cations are exemplified by sodium, potassium, calcium, magnesium, ammonium, tetraalkylammonium cations, and the like.

“Pharmaceutically acceptable vehicle” refers to a diluent, adjuvant, excipient or carrier with which a compound of the invention is administered.

“Preventing” or “prevention” refers to a reduction in risk of acquiring a disease or disorder (i.e., causing at least one of the clinical symptoms of the disease not to develop in a subject not yet exposed to or predisposed to the disease, and not yet experiencing or displaying symptoms of the disease).

“Prodrugs” refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like.

“Solvate” refers to forms of the compound that are associated with a solvent, usually by a solvolysis reaction. Conventional solvents include water, ethanol, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates.

“Subject” includes humans. The terms “human,” “patient” and “subject” are used interchangeably herein.

“Therapeutically effective amount” means the amount of a compound that, when administered to a subject for treating a disease or a condition, is sufficient to effect such treatment for the disease or condition. The “therapeutically effective amount” can vary depending on the compound, the disease or condition and its severity, and the age, weight, etc., of the subject to be treated.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder, or even preventing the same. In a still further embodiment, “treating” or “treatment” refers to administration of the compound or composition of the invention for cosmetic purposes.

Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Preferred are the Cᵢ to C₈ alkyl, C₂-C₈ alkenyl, aryl, C₇-C₁₂ substituted aryl, and C₇-C₁₂ arylalkyl esters of the compounds of the invention.

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (2H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy.

All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of it electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base.

Tautomeric forms may be relevant to the attainment of the optimal chemical reactivity and biological activity of a compound of interest.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

### The Compounds

As described herein, the present invention relates to the identification of compounds that control melanin synthesis (melanogenesis), and the use of such compounds and compositions thereof to modify (e.g., inhibit) melanin production. This invention also relates to methods for preventing and/or treating conditions that are causally related to aberrant melanogenesis activity, comprising (but not limited to) aberrant pigmentation, including hyperpigmentation of all etiologies, uneven pigmentation, and the like, using the compounds and compositions of the invention.

In accordance with the present invention, a plurality of compounds has been identified that are capable of controlling, and particularly, inhibiting melanogenesis. These compounds, which were not previously identified as possessing such a capability, are listed herein and referred to as novel melanogenesis modifiers. Accordingly, the present invention is directed to their use in modifying pigmentation in in vitro and in vivo applications. With respect to in vitro applications, test-tube based and additional cell-based assays may be used to test the ability of modified versions and/or derivatives of compounds listed herein to alter melanogenesis. In vivo applications are directed to the administration of at least one of the novel melanogenesis inhibiting compounds listed herein to a subject in need thereof to reduce pigmentation levels for prophylactic, therapeutic and/or cosmetic purposes.

Thus, in one aspect of the present invention, compounds have been identified that are capable of effectively and efficiently inhibiting melanogenesis (referred to herein as melanogenesis inhibitors) in mammalian cells. The ability of such compounds to decrease or inhibit melanogenesis may be used to advantage to decrease the melanin content of melanocytes, which, in turn, results in decreased pigmentation or lightening of skin, hair, wool, or fur color. In view of the above, the novel melanogenesis inhibitors of the present invention may be topically applied to skin, hair, wool, or fur to lighten their color.

In one embodiment of the invention, steroidal compounds are disclosed that are melanogenesis modifiers, such as inhibitors, having a formula (I):

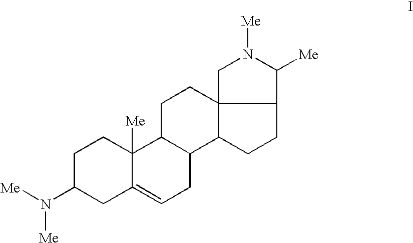

or a pharmaceutically acceptable salt, solvate or prodrug thereof, and stereoisomers, tautomers and isotopic variants thereof.

In one particular embodiment of the invention, with respect to formula I, the compound is conessine.

In another embodiment of the invention, steroidal compounds are disclosed that are melanogenesis modifiers, such as inhibitors. having a formula (II):

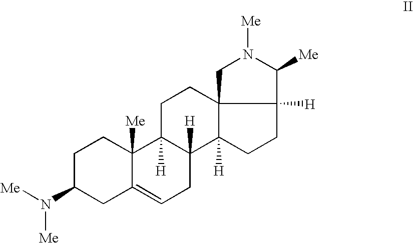

or a pharmaceutically acceptable salt, solvate or prodrug thereof, and stereoisomers, tautomers and isotopic variants thereof.

In a further embodiment of the invention, steroidal compounds are disclosed that are melanogenesis modifiers, such as inhibitors. having a formula (III):

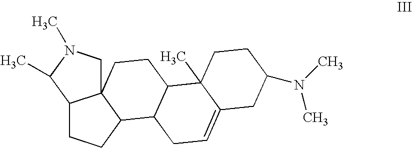

or a pharmaceutically acceptable salt, solvate or prodrug thereof, and stereoisomers, tautomers and isotopic variants thereof.

In one particular embodiment of the invention, with respect to formulae I-III, the compound is a free base.

In one particular embodiment of the invention, with respect to formulae I-III, the compound is a mono HCl salt.

In one particular embodiment of the invention, with respect to formulae I-III, the compound is a di HCl salt.

In one particular embodiment of the invention, with respect to formulae I-III, the compound is a hydrated salt.

A further aspect of the invention extends to a formulation that comprises a combination of a compound with respect to formulae I-III, and a like-acting agent. In a particular embodiment, the like acting agent is selected from a cosmetic ingredient and a pharmacologically active agent. More particularly, the cosmetic ingredient is a skin lightener, and the pharmacologically active agent is selected from another melanogenesis inhibitor.

In a particular embodiment of the combination just described, a pharmaceutical composition is prepared that is useful to treat a disease for which a melanogenesis inhibitor is indicated, which comprises a therapeutically effective amount of the combination, wherein the like acting agent is a pharmacologically active agent. More particularly, the like-acting agent is a skin lightening compound.

In a further embodiment of the combination described above, a topical formulation is prepared that comprises a composition for cosmetic or dermatological use, which composition comprises a cosmetically and/or dermatologically effective amount of the combination stated above, wherein the like acting agent is a cosmetically active agent. More particularly, the like-acting agent is a skin lightening compound.

In a further aspect of the invention, the invention extends to a method for preventing, treating, ameliorating or managing a disease or condition involving undesired or aberrant melanogenesis, which comprises administering to a patient in need or desirous of such prevention, treatment, amelioration or management, a pharmaceutical composition comprising a prophylactically or therapeutically effective melanogenesis-inhibiting amount of the combination as stated and set forth above, wherein the like acting agent is a pharmacologically active agent. More particularly, the like-acting agent is a skin lightening compound.

A method for altering or restoring pigmentation in mammalian skin, hair, wool or fur comprising administering to the mammalian skin, hair, wool or fur an amount of a composition comprising a pigment restoring or altering-effective amount of the combination as stated and set forth above, wherein the like acting agent is a cosmetically active agent. More particularly, the like-acting agent is a skin lightening compound.

The methods and compositions of the present invention contemplate the use of one or more of the compounds listed herein as an active ingredient(s) for various uses. In a particular embodiment, the active ingredient(s) is combined with an acceptable carrier to form a topical formulation for application to the skin, for example, for cosmetic and/or therapeutic dermatological uses. Topical formulations may include ointments, lotions, pastes, creams, gels, drops, suppositories, sprays, liquids, shampoos, powders and transdermal patches. Thickeners, diluents, emulsifiers, dispersing aids or binders may be used as needed. Preferably, one function of the carrier is to enhance skin penetration of the active ingredient(s), and should be capable of delivering the active ingredient(s) to melanocytes under in vivo conditions. Suitable carriers are well known to skilled practitioners, and include liposomes, ethanol, dimethylsulfoxide (DMSO), petroleum jelly (petrolatum), mineral oil (liquid petrolatum), water, dimethylformamide, dekaoxyethylene-oleylether, oleic acid, 2-pyrrolidone and Azone® brand penetration enhancer (Upjohn). A particular composition may be formulated to include an active ingredient(s) as described in Table I, with one of 2-pyrrolidone, oleic acid and/or Azone® added to enhance penetration, solubilized in a base of water, ethanol, propanol and/or propylene glycol.

As indicated above, vehicles comprising liposomes may be used for topical delivery of some of the compositions of the invention. Depending on the composition, and at the discretion of a skilled practitioner, such liposomes may be non-ionic and contain a) glycerol dilaurate (preferably in an amount of between about 5% and about 70% by weight); b) compounds having the steroid backbone found in cholesterol (preferably in an amount of between about 5% and about 45% by weight); and c) one or more fatty acid ethers having from about 12 to about 18 carbon atoms preferably in an amount of between about 5% and about 70% by weight collectively), wherein the constituent compounds of the liposomes are preferably in a ratio of about 37.5:12.5:33.3:16.7. For some compositions, liposomes comprised of glycerol dilaurate/cholesterol/polyoxyethylene-10-stearyl ether/polyoxyethylene-9-lauryl ether (GDL liposomes) are preferred. Liposomes may be present in an amount, based upon the total volume of the composition, of from about 10 mg/mL to about 100 mg/mL, and more preferably from about 20 mg/mL to about 50 mg/mL. A ratio of about 37.5:12.5:33.3:16.7 may be used to particular advantage. Suitable liposomes may be prepared in accordance with standard methods commonly used in the art.

The above described composition may be prepared by combining the desired components in a suitable container and mixing them under ambient conditions in any conventional high shear mixing means well known in the art for non-ionic liposome preparations, such as those disclosed in Niemiec et al. (Pharm. Res. 12:1184-88 (1995)), which is incorporated by reference herein in its entirety. The presence of such liposomes enhances the depigmenting capabilities of some compositions.

Other formulations may contain, for example, soybean milk or other liquid formulations derived directly from legumes or other suitable plant. Such a formulation may, for example, contain a large proportion of soybean milk, an emulsifier that maintains the physical stability of the soybean milk, and, optionally a chelating agent, preservatives, emollients, humectants and/or thickeners or gelling agents.

Oil-in-water emulsions, water-in-oil emulsions, solvent-based formulations and aqueous gels known to those of skill in the art may also be utilized as vehicles for the delivery of the compositions of this invention.

Depending on the specific application, the compositions of the present invention may also include other active ingredients, as well as inert or inactive ingredients. In such alternative embodiments, the topically active pharmaceutical or cosmetic composition may be optionally combined with other ingredients such as moisturizers, cosmetic adjuvants, surfactants, foaming agents, conditioners, humectants, fragrances, viscosifiers, buffering agents, preservatives, sunscreens and the like.

Particular formulations may include at least one active ingredient (for example, a novel melanogenesis modifier described herein) or previously recognized, and particularly, like-acting agents, such as skin lighteners and skin-brighteners, which may be known to those of skill in the art. Agents known to possess similar activities and/or properties include, but are not limited to: bleaching agents; tyrosinase inhibitors; α-hydroxy acids, salts and derivatives thereof; α-keto acids, salts and derivatives thereof; β-hydroxy acids, salts and derivatives thereof; retinoids, salts and derivatives thereof; Vitamin A and related compounds; acids; phenol; methoxypropyl-gluconamide; corticosteroids; agents that block the transfer of melanosomes to keratinocytes, such as may be found in soy extracts; kojic acid; licorice extracts; and the like.

Additional non-limiting examples of skin-lightening and brightening agents include kojic acid (5-hydroxy-2-hydroxymethyl-4-pyranone), kojic acid derivatives e.g. kojic acid dipalmitate, arbutin, deoxyarbutin, ascorbic acid, ascorbic acid derivatives, hydroquinone, hydroquinone derivatives, resorcinol, sulfur-containing molecules such as e.g. glutathione or cysteine, alpha-hydroxy acids (e.g. citric acid, lactic acid, malic acid) and derivatives thereof, N-acetyl tyrosine and derivatives, undecenoyl phenylalanine, gluconic acid, 4-alkyl resorcinols, 4-(1-phenylethyl)-1,3-dihydroxybenzene, chromone derivatives such as aloesin, flavonoids, thymol derivatives, 1-aminoethyl phosphinic acid, thiourea derivatives, ellagic acid, nicotinamide (niacinamide), zinc salts such as e.g. zinc chloride or gluconate, thujaplicin and derivatives, triterpenes such as maslinic acid, sterols such as ergosterol, benzofuranones such as senkyunolide, vinyl and ethyl guiacol, dionic acids such as octodecene dionic acid and azelaic acid, inhibitors of nitrogen oxide synthesis, such as e.g. L-nitroarginine and derivatives thereof, 2,7 dinitroindazole or thiocitrulline, metal chelators (e.g. alpha-hydroxy fatty acids, palmitic acid, phytic acid, lactoferrin, humic acid, bile acid, bile extracts, bilirubin, biliverdin, EDTA, EGTA and derivatives thereof), retinoids, soya milk and extract, serine protease inhibitors or lipoic acid or other synthetic or natural active ingredients for skin and hair lightening, the latter also being used in the form of an extract from plants, such as e.g. bearberry extract, rice extract, papaya extract, and licorice root extract; or constituents concentrated from such extracts, such as glabridin or Iicochalcone A, artocarpus extract, extract of rumex and ramulus species, extracts of pine species (pinus) and extracts of vitis species or stilbene derivatives concentrated therefrom, extract of saxifrage, mulberry, scutelleria or/and grapes.

The amount of the aforementioned examples of additional active ingredients for skin and hair lightening or brightening (one or more compounds) in the formulations according to the invention may be from about 0.01 to about 30 wt. %, particularly from about 0.01 to about 20 wt. %, and more particularly from about 0.01 to about 5 wt. %, based on the total weight of the preparation. The foregoing ranges are presented by way of illustration and not limitation.

The dose regimen will depend on a number of factors which may readily be determined, such as severity and responsiveness of the condition to be treated, but will normally be one or more doses per day, with a course of treatment lasting from several days to several months, or until a cure is effected or a diminution of disease state is achieved, or a cosmetically desired degree of melanogenesis modification (e.g., reduction in pigmentation) is achieved, depending on the application. One of ordinary skill may readily determine optimum dosages, dosing methodologies and repetition rates. In general, it is contemplated that topical formulations (such as creams, lotions, solutions, etc.) will have a concentration of active ingredient of from about 0.01% to about 50%, preferably from about 0.1% to about 10%. In general, it is contemplated that unit dosage form compositions according to the present invention will contain from about 0.01 mg to about 100 mg of active ingredient, preferably about 0.1 mg to about 10 mg of active ingredient.

In general, melanogenesis inhibitors or compounds that decrease or suppress melanin production and pigmentation in mammalian skin, hair, fur or wool are useful in, for example, the lightening and/or brightening of skin, hair, wool or fur for cosmetic purposes, or the treatment of hyperpigmentation or uneven pigmentation disorders such as vitiligo, ephelides, lentigines, dermal melanocytosis, cafe-au-lait spots, post-inflammatory hyperpigmentation, etc. For such depigmentation applications, the formulation and dosing would be as described above.

In certain aspects, the present invention provides prodrugs and derivatives of the compounds of the invention. Prodrugs are derivatives of the compounds of the invention, which have metabolically cleavable groups and become by solvolysis or under physiological conditions the compounds of the invention, which are pharmaceutically active, in vivo. Such examples include, but are not limited to, choline ester derivatives and the like, N-alkylmorpholine esters and the like.

Other derivatives of the compounds of this invention have activity in both their acid and acid derivative forms, but the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well know to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are preferred prodrugs. In some cases it is desirable to prepare double ester type prodrugs such as (acyloxy)alkyl esters or ((alkoxycarbonyl)oxy)alkylesters. Preferred are the Cᵢ to C₈ alkyl, C₂-Cₛ alkenyl, aryl, C₇-C₁₂ substituted aryl, and C₇-C₁₂ arylalkyl esters of the compounds of the invention.

The present invention also relates to the pharmaceutically acceptable acid addition and base salts of any of the aforementioned compounds of formulae I-III. The acids which are used to prepare the pharmaceutically acceptable acid addition salts of the aforementioned base compounds of this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate (i.e., 1,1-methylene-bis-(2-hydroxy-3-naphthoate)) salts.

The compounds useful according to the invention that are basic in nature are capable of forming a wide variety of different salts with various inorganic and organic acids. Although such salts must be pharmaceutically acceptable for administration to animals, it is often desirable in practice to initially isolate a compound of formula I from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the active base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained.

Those compounds useful according to the invention that are acidic in nature are capable of forming base salts with various pharmaceutically acceptable cations. Examples of such salts include the alkali metal and alkaline earth metal salts and, particularly, the sodium and potassium salts. These salts can be prepared by conventional techniques. The chemical bases that are used as reagents to prepare the pharmaceutically acceptable base salts of this invention are those that form non-toxic base salts with the acidic compounds of formula I. Such non-toxic base salts include those derived from such pharmaceutically acceptable cations as sodium, potassium, calcium and magnesium, etc. These salts can easily be prepared by treating the corresponding acidic compounds with an aqueous solution containing the desired pharmaceutically acceptable cations, and then evaporating the resulting solution to dryness, preferably under reduced pressure. Alternatively, they can also be prepared by mixing lower alkanolic solutions of the acidic compounds and the desired alkali metal alkoxide together, and then evaporating the resulting solution to dryness, as described above. In either case, stoichiometric quantities of reagents are preferably employed in order to ensure completeness of reaction and maximum yields of the desired final products.

The compounds useful according to the invention, and their pharmaceutically acceptable salts, are useful in the treatment of disorders of human pigmentation, including solar and simple lentigines (including age/liver spots), melasma/chloasma and postinflammatory hyper-pigmentation. Such compounds reduce skin melanin levels by inhibiting the production of melanin, whether the latter is produced constitutively or in response to UV irradiation (such as sun exposure). Thus, some of the active compounds used in this invention can be used to reduce skin melanin content in non-pathological states so as to induce a lighter skin tone, as desired by the user, or to prevent melanin accumulation in skin that has been exposed to UV irradiation. They can also be used in combination with skin peeling agents (including glycolic acid or trichloroacetic acid face peels) to lighten skin tone and prevent repigmentation.

The appropriate dose regimen, the amount of each dose administered, and specific intervals between doses of the active compound will depend upon the particular active compound employed, the condition of the patient being treated, and the nature and severity of the disorder or condition being treated. Preferably, the active compound is administered in an amount and at an interval that results in the desired treatment of or improvement in the disorder or condition being treated.

For skin lightening, an active compound used in the present invention can also be used in combination with sun screens (UVA or UVB blockers) to prevent repigmentation, to protect against sun or UV-induced skin darkening or to enhance their ability to reduce skin melanin and their skin bleaching action. For skin lightening, an active compound used in the present invention can also be used in combination with retinoic acid or its derivatives or any compounds that interact with retinoic acid receptors and accelerate or enhance the invention's ability to reduce skin melanin and skin bleaching action, or enhance the invention's ability to prevent the accumulation of skin melanin. For skin lightening, an active compound used in the present invention can also be used in combination with 4-hydroxyanisole. For skin lightening, the active compounds used in this invention can also be used in combination with ascorbic acid, its derivatives and ascorbic-acid based products (such as magnesium ascorbate) or other products with an anti-oxidant mechanism (such as resveratrol) which accelerate or enhance their ability to reduce skin melanin and their skin bleaching action.

The invention also provides compounds useful to decrease melanin production or to reduce skin pigmentation, which correspond to compounds of the formulae I-III, and prodrugs, and analogs thereof, and to pharmaceutical compositions containing them, and including any pharmaceutically acceptable salts or solvates thereof.

Although methods and materials similar or equivalent to those described herein can be used in the practice or testing of the invention, preferred methods and materials are described below. The materials, methods, and examples are illustrative only and not intended to be limiting. Other features and advantages of the invention will be apparent from the detailed description, examples, and the claims.

### Rational Drug Design

Compounds identified by the methods of the invention or compounds disclosed herein may serve as the basis for molecular modeling techniques for the design of chemical analogs that are more effective. For example, chemical analogs of any of the compounds listed herein can be created using these or other modeling techniques. Examples of molecular modeling systems are the CHARM (Polygen Corporation, Waltham, Mass.) and QUANTA (Molecular Simulations Inc., San Diego, Calif.) programs. CHARM performs the energy minimization and molecular dynamics functions. QUANTA performs the construction, graphic modeling and analysis of molecular structure. QUANTA allows interactive construction, modification, visualization, and analysis of the behavior of molecules with each other.

For example, compounds of the present invention can further be used to design more effective analogs using modeling packages such as Ludi, Insight II, C²-Minimizer and Affinity (Molecular Simulations Inc., San Diego, Calif.). A particularly preferred modeling package is MacroModel (Columbia University, New York, N.Y.).

The compounds of the present invention can further be used as the basis for developing a rational combinatorial library. Such a library can also be screened to identify more effective compounds. While the nature of the combinatorial library is dependent on various factors such as the particular compound chosen from the preferred compounds of the present invention to form the basis of the library, as well as the desire to synthesize the library using a resin, it will be recognized that the compounds of the present invention provide requisite data suitable for combinatorial design programs such as C²-QSAR (Molecular Simulations Inc., San Diego, Calif.).

### Methods of Inhibiting Melanogenesis

As stated above, the compounds of the invention can be used to treat animals or, preferably, humans that have diseases, conditions, or disorders caused by the production or overproduction of melanin. Such diseases, conditions, or disorders include those that can be characterized by discolorations of the skin or hair such as, for example, hyperpigmentation caused by inflammation or from diseases such as melasma, or brown spots such as “cafe au lait” macules. Alternatively, a subject may wish to lighten the color of his or her hair or skin.

For the purposes of this application, the terms “treatment”, “therapeutic use”, and “medicinal use” shall refer to any and all uses of the compositions of the invention which remedy a disease state or one or more symptoms, or otherwise prevent, hinder, retard, or reverse the progression of disease or one or more other undesirable symptoms in any way whatsoever.

The invention further provides methods and pharmaceutical compositions for inhibiting skin pigmentation comprising the use of the present compounds either alone or in combination with other, like-acting agents, For example, such additional agents may include agents that are believed to function by mimicking or increasing P-protein function, and/or by modifying late endosomal/lysosomal trafficking. Such pharmaceutical compositions and their corresponding methods are useful for decreasing and/or inhibiting melanin production and, therefore, for reducing skin pigmentation. These agents may be used singly, in combination with one another, or in combination with other drugs that inhibit pigmentation. By way of a non-limiting example, other drugs that inhibit pigmentation include agents such as tyrosinase inhibitors. Preferably, the methods and compositions of the invention are for application to a vertebrate, more particularly to a mammal, and most preferably to a human.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent.

By the phrase “decrease in melanin production” is meant a detectable lowering of the amount of melanin synthesized de novo by a melanocyte exposed to a compound of the invention, as compared with the amount of melanin synthesized de novo by a control, untreated melanocyte. The term “lowering” as presently used refers, in a first instance, to a decrease of at least about 10%, in a further instance, to a decrease of at least about 25%, and in a still further instance, to a decrease of at least about 50%, in the amount of melanin synthesized de novo.

The term “late endosomal/lysosomal trafficking” is used herein to refer to the movement of proteins, lipids, or other compounds between different cellular compartments. These locations include the movement of such compounds from the late endosome to the lysosome, from the lysosome to the late endosome, from the late endosome or lysosome to the trans Golgi network, and from the trans Golgi network to the late endosome or lsysome.

An alteration in late endosomal/lysosomal trafficking may be effected by contacting the melanocyte with a compound such as progesterone, a hydrophobic amine, sphingosine, an antagonist of late endosomal/lysosomal trafficking, or any of the compounds of the formulae (I)-(IV) as set forth herein.

As one skilled in the art would know in view of this disclosure, the compounds used in the methods of the present invention may be used alone or in combination with each other. Moreover, the methods of the invention also include the additional use of other compounds known in the art to affect melanin synthesis such as tyrosinase inhibitors. Such inhibitors are known to those skilled in the art and include various derivatives of resorcinol, hydroquinone, kojic acid, melamine, and various types of plant extracts, among others.

Thus, the invention relates both to methods of modifying, and particularly inhibiting the pigmentation of skin in which the active compound used according to the invention, or a pharmaceutically acceptable salt thereof, and one or more of the other active ingredients referred to above are administered together, as part of the same pharmaceutical composition, as well as methods in which they are administered separately as part of an appropriate dose regimen designed to obtain the benefits of the combination therapy. The appropriate dose regimen, the amount of each dose administered, and specific intervals between doses of each active agent will depend upon the specific combination of active agents employed, the condition of the patient being treated, and the nature and severity of the disorder or condition being treated. Such additional active ingredients will generally be administered in amounts less than or equal to those for which they are effective as single topical therapeutic agents. The FDA approved dosages for such active agents that have received FDA approval for administration to humans are publicly available.

For example, any of the compounds used according to a skin-lightening method of the invention may be used in combination with a tyrosinase inhibitor or other skin-lightening and/or skin brightening agent as currently known in the art or to be developed in the future, including any one or more of those agents described in the following patent publications: U.S. Pat. No. 4,278,656 to Nagai et al, issued Jul. 14, 1981; U.S. Pat. No. 4,369,174 to Nagai et al., issued Jan. 18, 1983; U.S. Pat. No. 4,959,393 to Torihara et al., issued Sep. 25, 1990; U.S. Pat. No. 5,580,549 to Fukuda et al., issued Dec. 3, 1996; U.S. Pat. No. 6,123,959 to Jones et al., issued Sep. 26, 2000; U.S. Pat. No. 6,132,740 to Hu, issued Oct. 17, 2000; U.S. Pat. No. 6,159,482 to Tuloup et al., issued Dec. 12, 2000; WO 99/32077 by L'Oreal, published Jul. 1, 1999; WO 99/64025 by Fytokem Prod. Inc., published Dec. 16, 1999; WO 00/56702 by Pfizer Inc., published Sep. 28, 2000; WO 00/76473 by Shiseido Co. Ltd., published Dec. 12, 2000; EP 997140 by L'Oreal SA, published May 3, 2000; JP 5221846 by Kunimasa Tomoji, published Aug. 31, 1993; JP 7242687 by Shiseido Co. Ltd., published Sep. 19, 1995; JP 7324023 by Itogawa H, published Dec. 12, 1995; JP 8012552 by Shiseido Co. Ltd., published Jan. 16, 1996; JP 8012554 by Shiseido Co. Ltd., published Jan. 16, 1996; JP 8012557 by Shiseido Co. Ltd., published Jan. 16, 1996; JP 8012560 by Shiseido Co. Ltd., published Jan. 16, 1996; JP 8012561 by Shiseido Co. Ltd., published Jan. 16, 1996; JP 8134090 by Fujisawa, published May 28, 1996; JP 8168378 by Kirinjo KK, published Jul. 2, 1996; JP 8277225 by Kansai Koso KK, published Oct. 22, 1996; JP 9002967 by Sanki Shoji KK, published Jan. 7, 1997; JP 9295927 by Yagi Akira, published Nov. 18, 1997; JP 10072330 by Kansai Kouso, published Mar. 17, 1998; JP 10081626 by Kamiyama KK, published Mar. 31, 1998; JP 10101543 by Kansai Kouso KK, published Apr. 21, 1998; JP 11071231 by Maruzen Pharm., published Mar. 16, 1999; JP 11079934 by Kyodo Nyugyo, published Mar. 23, 1999; JP 11246347 by Shiseido Co. Ltd., published Sep. 14, 1999; JP 11246344 by Shiseido Co. Ltd., published Sep. 14, 1999; JP 2000-080023 by Kanebo Ltd., published Mar. 21, 2000; JP 2000-095663 by Kose KK, published Apr. 4, 2000; JP 2000-159681 by Hai Tai Confectionary Co. Ltd., published Jun. 13, 2000; JP 2000-247907 by Kanebo Ltd., published Sep. 12, 2000; JP-9002967 by Sanki Shoji KK, published Jan. 7, 1997; JP-7206753 by Nikken Food KK, published Aug. 8, 1995; JP-5320025 by Kunimasa T, published Dec. 3, 1993; JP-59157009 by Yakurigaku Chuou KE, published Sep. 6, 1984, and WO 2007/110415 by Symrise GmbH & Co., KG, published Oct. 4, 2007; among others; which patent publications are incorporated herein by reference in their entireties.

Non-limiting examples of compounds that cause an alteration in late endosomal/lysosomal trafficking include the compounds of formulae I-III herein, either alone or in combination with each other. Particular derivatives have been described herein.

In another aspect, the invention provides a method of reducing skin pigmentation. In this method the skin is contacted with a pharmaceutically effective amount of a compound that effects an alteration in late endosomal/lysosomal trafficking, wherein an alteration in late endosomal/lysosomal trafficking results in a reduction of skin pigmentation.

By the phrase “reducing skin pigmentation” is meant a detectable decrease in the amount of melanin in the skin, preferably causing a lightening of the skin as a result of a lowering of the amount of melanin synthesized de novo. The term “lowering” as presently used refers, in a first instance, to a decrease of at least about 10%, in a further instance, to a decrease of at least about 25%, and in a still further instance, to a decrease of at least about 50%, in the amount of melanin synthesized de novo. This lowering of melanin synthesized de novo is preferably visually distinguishable to the naked eye, i.e., would not require the aid of a microscope or other such means to detect its occurrence.

The invention also provides for a reduction in skin pigmentation by contacting the skin topically with an effective amount of a compound that alters late endosomal/lysosomal trafficking in the skin. Useful compounds for these methods of the invention include those disclosed above.

### Pharmaceutical Applications

For pharmaceutical uses, it is preferred that the compounds of the invention are part of a pharmaceutical composition. Pharmaceutical compositions, comprising an effective amount of such a compound in a pharmaceutically acceptable carrier, can be administered to a patient, person, or animal having a disease, disorder, or condition which is of a type that produces, or overproduces, melanin.

The amount of compound which will be effective in the treatment of a particular disease, disorder, or condition will depend on the nature of the disease, disorder, or condition, and can be determined by standard clinical techniques. Where possible, it is desirable to determine in vitro the cytotoxicity of the compound to the tissue type to be treated, and then in a useful animal model system prior to testing and use in humans.

The compound can be administered for the reduction or increase of melanin synthesis by any means that results in contact of the active agent with its site of action in the body of a mammal. The compounds can be administered by any conventional means available for use in conjunction with pharmaceuticals, either as individual therapeutic agents or in a combination of therapeutic agents. Each can be administered alone, but is preferably administered with a pharmaceutical carrier selected on the basis of the chosen route of administration and standard pharmaceutical practice. The pharmaceutical compositions of the invention can be adapted for oral, parenteral, rectal, and preferably topical, administration, and can be in unit dosage form, in a manner well known to those skilled in the pharmaceutical art. Parenteral administration includes but is not limited to, injection subcutaneously, intravenously, intraperitoneally or intramuscularly. However, topical application is preferred.

### Cosmetic Applications

In addition to pharmaceutical uses, the methods of the current invention are useful for cosmetic purposes. Cosmetic applications for methods of the present invention include the topical application of compositions containing one or more compounds to enhance or otherwise alter the visual appearance of skin or hair. Occurrences in the skin or hair of noticeable but undesired pigmentation as a result of melanin production, overproduction or underproduction can be treated using the methods of the present invention. Thus, and as discussed above, the compounds and compositions of the invention can be used to achieve improvements in skin or hair appearance, as by brightening the same, adding or enhancing luster, and the like. Suitable formulations for these purposes can be prepared by those skilled in the art, and such details of preparation are considered within the scope of the present invention.

### Endpoints and Dosages

An effective dosage and treatment protocol can be determined by conventional means, starting with a low dose in laboratory animals and then increasing the dosage while monitoring the effects, and systematically varying the dosage regimen as well. Animal studies, preferably mammalian studies, are commonly used to determine the maximal tolerable dose, or MTD, of a bioactive agent per kilogram weight. Those skilled in the art can extrapolate doses for efficacy and avoidance of toxicity to other species, including humans.

Before human studies of efficacy are undertaken, Phase I clinical studies in normal subjects can help establish safe doses. Numerous factors can be taken into consideration by a clinician when determining an optimal dosage for a given subject. Primary among these is the toxicity and half-life of the chosen compound that affects or mimics P protein function or that inhibits late endosomal/lysosomal trafficking Additional factors include the size of the patient, the age of the patient, the general condition of the patient, the particular disease, condition, or disorder being treated, the severity of the disease, condition, or disorder being treated, the presence of other drugs in the patient, the effect desired, and the like. The trial dosages would be chosen after consideration of the results of animal studies and the clinical literature.

One of ordinary skill in the art will appreciate that the endpoint chosen in a particular case will vary according to the disease, condition, or disorder being treated, the outcome desired by the patient, subject, or treating physician, and other factors. Where the composition is being used for cosmetic purposes, such as to lighten skin color such as, for example, to reverse hyperpigmentation caused by, for example, inflammation or diseases such as melasma, or to modify hair color, any one of a number of endpoints can be chosen.

In such instance, endpoints can be defined subjectively such as, for example, when the subject is simply “satisfied” with the results of the treatment. For pharmacological applications, the endpoint can be determined by the patient's, or the treating physicians, satisfaction with the results of the treatment. Alternatively, endpoints can be defined objectively. For example, the patient's or subject's skin or hair in the treated area can be compared to a color chart. Treatment is terminated when the color of the skin or hair in the treated area is similar in appearance to a color on the chart. Alternatively, the reflectance of the treated skin or hair can be measured, and treatment can be terminated when the treated skin or hair attains a specified reflectance. Alternatively, the melanin content of the treated hair or skin can be measured. Treatment can be terminated when the melanin content of the treated hair or skin reaches a specified value. Melanin content can be determined in any way known to the art, including by histological methods, with or without enhancement by stains for melanin.

### Methods of Administration

The compounds of the invention can be administered topically, e.g., as patches, ointments, creams, gels, lotions, solutions, foams, masks or transdermal administration. The compounds can also be administered orally in solid or semi-solid dosage forms, such as hard or soft-gelatin capsules, tablets, or powders, or in liquid dosage forms, such as elixirs, syrups, or suspensions. Additionally, the compounds can also be administered parenterally, in sterile liquid dosage forms or in suppository form.

Because in vivo use is contemplated, the composition is preferably of high purity and substantially free of potentially harmful contaminants, e.g., at least National Food (NF) grade, generally at least analytical grade, and preferably at least pharmaceutical grade. To the extent that a given compound must be synthesized prior to use, such synthesis or subsequent purification shall preferably result in a product that is substantially free of any potentially contaminating toxic agents that may have been used during the synthesis or purification procedures.

Useful pharmaceutical dosage forms for administration of the present compounds are described below.

The pharmaceutical compositions can be applied directly to the skin. Alternatively, they can be delivered by various transdermal drug delivery systems, such as transdermal patches as known in the art. For example, for topical administration, the active ingredient can be formulated in a solution, gel, lotion, ointment, cream, suspension, foam, mask, paste, liniment, powder, tincture, aerosol, patch, or the like in a pharmaceutically or cosmetically acceptable form by methods well known in the art. The composition can be any of a variety of forms common in the pharmaceutical or cosmetic arts for topical application to animals or humans, including solutions, lotions, sprays, creams, ointments, salves, gels, etc., as described below. Preferred agents are those that are viscous enough to remain on the treated area, those that do not readily evaporate, and/or those that are easily removed by rinsing with water, optionally with the aid of soaps, cleansers and/or shampoos. Actual methods for preparing topical formulations are known or apparent to those skilled in the art, and are described in detail in Remington's Pharmaceutical Sciences, 1990 (supra); and Pharmaceutical Dosage Forms and Drug Delivery Systems, 6th ed., Williams & Wilkins (1995).

In order to enhance the percutaneous absorption of the active ingredients, one or more of a number of agents can be added in the topical formulations including, but not limited to, dimethylsulfoxide, dimethylacetamide, dimethylformamide, surfactants, azone, alcohol, acetone, propylene glycol and polyethylene glycol. In addition, physical methods can also be used to enhance transdermal penetration such as, e.g., by iontophoresis or sonophoresis. Alternatively, or in addition, liposomes may be employed.

A topically applied composition of the invention contains a pharmaceutically effective amount of at least one of the compounds of the invention as described herein, and those ingredients as are necessary for use as a carrier, such as an emulsion, a cream, an ointment, an ophthalmic ointment, an aqueous solution, a lotion or an aerosol. Non-limiting examples of such carriers are described in more detail below and may be found in International Pat. Publication WO 00/62742, published Oct. 26, 2000, U.S. Pat. No. 5,691,380 to Mason et al., issued on Nov. 25, 1997 and U.S. Pat. No. 5,968,528 to Deckner et al., issued on Oct. 19, 1999, U.S. Pat. No. 4,139,619 to Chidsey, III, issued on Feb. 13, 1979 and U.S. Pat. No. 4,684,635 to Orentreich et al., issued on Aug. 4, 1987 which are incorporated herein by reference. Suitable pharmaceutical carriers are further described in Remington's Pharmaceutical Sciences, 17th ed., Mack Publishing Company, Easton, Pa. (1990) a standard reference text in this field.

The pharmaceutical compositions of the invention may also include optional components. Such optional components should be suitable for application to keratinous tissue, that is, when incorporated into the composition, they are suitable for use in contact with human keratinous tissue without undue toxicity, incompatibility, instability, allergic response, and the like within the scope of sound medical judgment. In addition, such optional components are useful provided that they do not unacceptably alter the benefits of the active compounds of the invention. The CTFA Cosmetic Ingredient Handbook, Second Edition (1992) describes a wide variety of non-limiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyffhizinate), skin treating agents, thickeners, and vitamins and derivatives thereof.

In addition to the pharmaceutically effective amount of an agent disclosed herein, the topical compositions of the present invention also comprise a dermatologically acceptable carrier. The phrase “dermatologically acceptable carrier”, as used herein, means that the carrier is suitable for topical application to the skin, i.e., keratinous tissue, has good aesthetic properties, is compatible with the active agents of the present invention and any other components, and will not cause any safety or toxicity concerns. A safe and effective amount of carrier is from about 50% to about 99.99%, preferably from about 80% to about 99.9%, more preferably from about 90% to about 98%, and most preferably from about 90% to about 95% of the composition.

The carrier utilized in the compositions of the invention can be in a wide variety of forms. These include emulsion carriers, including, but not limited to, oil-in-water, water-in-oil, water-in-oil-in-water, and oil-in-water-in-silicone emulsions, a cream, an ointment, an ophthalmic ointment, an aqueous solution, a lotion or an aerosol. As will be understood by the skilled artisan, a given component will distribute primarily into either the water or oil/silicone phase, depending on the water solubility/dispersibility of the component in the composition.

Emulsions according to the present invention generally contain a pharmaceutically effective amount of an agent disclosed herein and a lipid or oil. Lipids and oils may be derived from animals, plants, or petroleum and may be natural or synthetic (i.e., man-made). Preferred emulsions also contain a humectant, such as glycerin. Emulsions will preferably further contain from about 1% to about 10%, more preferably from about 2% to about 5%, of an emulsifier, based on the weight of the carrier. Emulsifiers may be nonionic, anionic or cationic. Suitable emulsifiers are described in, for example, U.S. Pat. No. 3,755,560, issued to Dickert, et al. Aug. 28, 1973; U.S. Pat. No. 4,421,769, issued to Dixon, et al. Dec. 20, 1983; and McCutcheon's Detergents and Emulsifiers, North American Edition, pages 317-324 (1986).

The emulsion may also contain an anti-foaming agent to minimize foaming upon application to the keratinous tissue. Anti-foaming agents include high molecular weight silicones and other materials well known in the art for such use.

Suitable emulsions may have a wide range of viscosities, depending on the desired product form. Exemplary low viscosity emulsions, which are preferred, have a viscosity of about 50 centistokes or less, more preferably about 10 centistokes or less, most preferably about 5 centistokes or less. The emulsion may also contain an anti-foaming agent to minimize foaming upon application to the keratinous tissue. Anti-foaming agents include high molecular weight silicones and other materials well known in the art for such use.

One type of emulsion is a water-in-silicone emulsion. Water-in-silicone emulsions contain a continuous silicone phase and a dispersed aqueous phase. Preferred water-in-silicone emulsions of the present invention comprise from about 1% to about 60%, preferably from about 5% to about 40%, more preferably from about 10% to about 20%, by weight of a continuous silicone phase. The continuous silicone phase exists as an external phase that contains or surrounds the discontinuous aqueous phase described hereinafter.

The continuous silicone phase may contain a polyorganosiloxane oil. A preferred water-in-silicone emulsion system is formulated to provide an oxidatively stable vehicle for delivery of a pharmaceutically effective amount of an agent disclosed herein. The continuous silicone phase of these preferred emulsions comprises between about 50% and about 99.9% by weight of organopolysiloxane oil and less than about 50% by weight of a non-silicone oil. In an especially preferred embodiment, the continuous silicone phase comprises at least about 50%, preferably from about 60% to about 99.9%, more preferably from about 70% to about 99.9%, and even more preferably from about 80% to about 99.9%, polyorganosiloxane oil by weight of the continuous silicone phase, and up to about 50% non-silicone oils, preferably less about 40%, more preferably less than about 30%, even more preferably less than about 10%, and most preferably less than about 2%, by weight of the continuous silicone phase. These useful emulsion systems may provide more oxidative stability over extended periods of time than comparable water-in-oil emulsions containing lower concentrations of the polyorganosiloxane oil. Concentrations of non-silicone oils in the continuous silicone phase are minimized or avoided altogether so as to possibly further enhance oxidative stability of the active compound of the invention in the compositions. Water-in-silicone emulsions of this type are described in U.S. Pat. No. 5,691,380 to Mason et al., issued Nov. 25, 1997.

The organopolysiloxane oil for use in the composition may be volatile, non-volatile, or a mixture of volatile and non-volatile silicones. The term “nonvolatile” as used in this context refers to those silicones that are liquid under ambient conditions and have a flash point (under one atmospheric of pressure) of or greater than about 100° C. The term “volatile” as used in this context refers to all other silicone oils. Suitable organopolysiloxanes can be selected from a wide variety of silicones spanning a broad range of volatilities and viscosities. Examples of suitable organopolysiloxane oils include polyalkylsiloxanes, cyclic polyalkylsiloxanes, and polyalkylarylsiloxanes, which are known to those skilled in the art and commercially available.

The continuous silicone phase may contain one or more non-silicone oils. Concentrations of non-silicone oils in the continuous silicone phase are preferably minimized or avoided altogether so as to further enhance oxidative stability of the pharmaceutically effective agent in the compositions. Suitable non-silicone oils have a melting point of about 25.degree. C. or less under about one atmosphere of pressure. Examples of non-silicone oils suitable for use in the continuous silicone phase are those well known in the chemical arts in topical personal care products in the form of water-in-oil emulsions, e.g. mineral oil, vegetable oils, synthetic oils. semisynthetic oils, etc.

Useful topical compositions of the present invention comprise from about 30% to about 90%, more preferably from about 50% to about 85%, and most preferably from about 70% to about 80% of a dispersed aqueous phase. In emulsion technology, the term “dispersed phase” is a term well-known to one skilled in the art which means that the phase exists as small particles or droplets that are suspended in and surrounded by a continuous phase. The dispersed phase is also known as the internal or discontinuous phase. The dispersed aqueous phase is a dispersion of small aqueous particles or droplets suspended in and surrounded by the continuous silicone phase described hereinbefore. The aqueous phase can be water, or a combination of water and one or more water soluble or dispersible ingredients. Nonlimiting examples of such optional ingredients include thickeners, acids, bases, salts, chelants, gums, water-soluble or dispersible alcohols and polyols, buffers, preservatives, sunscreening agents, colorings, and the like.

The topical compositions of the present invention typically comprise from about 25% to about 90%, preferably from about 40% to about 80%, more preferably from about 60% to about 80%, water in the dispersed aqueous phase by weight of the composition.

The water-in-silicone emulsions of the present invention preferably comprise an emulsifier. In a preferred embodiment, the composition contains from about 0.1% to about 10% emulsifier, more preferably from about 0.5% to about 7.5%, most preferably from about 1% to about 5%, emulsifier by weight of the composition. The emulsifier helps disperse and suspend the aqueous phase within the continuous silicone phase.

A wide variety of emulsifying agents can be employed herein to form the preferred water-in-silicone emulsion. Known or conventional emulsifying agents can be used in the composition, provided that the selected emulsifying agent is chemically and physically compatible with essential components of the composition, and provides the desired dispersion characteristics. Suitable emulsifiers include silicone emulsifiers, e.g., organically modified organopolysiloxanes, also known to those skilled in the art as silicone surfactants, non-silicon-containing emulsifiers, and mixtures thereof, known by those skilled in the art for use in topical personal care products.

Useful emulsifiers include a wide variety of silicone emulsifiers. These silicone emulsifiers are typically organically modified organopolysiloxanes, also known to those skilled in the art as silicone surfactants. Suitable emulsifiers are described, for example, in McCutcheon's, Detergents and Emulsifiers, North American Edition (1986), published by Allured Publishing Corporation; U.S. Pat. No. 5,011,681 to Ciotti et al., issued Apr. 30, 1991; U.S. Pat. No. 4,421,769 to Dixon et al., issued Dec. 20, 1983; and U.S. Pat. No. 3,755,560 to Dickert et al., issued Aug. 28, 1973.

Other preferred topical carriers include oil-in-water emulsions, having a continuous aqueous phase and a hydrophobic, water-insoluble phase (“oil phase”) dispersed therein. Examples of suitable carriers comprising oil-in-water emulsions are described in U.S. Pat. No. 5,073,371 to Turner, D. J. et al., issued Dec. 17, 1991, and U.S. Pat. No. 5,073,372, to Turner, D. J. et al., issued Dec. 17, 1991. An especially preferred oil-in-water emulsion, containing a structuring agent, hydrophilic surfactant and water, is described in detail hereinafter.

A preferred oil-in-water emulsion comprises a structuring agent to assist in the formation of a liquid crystalline gel network structure. Without being limited by theory, it is believed that the structuring agent assists in providing rheological characteristics to the composition which contribute to the stability of the composition. The structuring agent may also function as an emulsifier or surfactant. Preferred compositions of this invention comprise from about 0.5% to about 20%, more preferably from about 1% to about 10%, most preferably from about 1% to about 5%, by weight of the composition, of a structuring agent. The preferred structuring agents of the present invention are selected from the group consisting of stearic acid, palmitic acid, stearyl alcohol, cetyl alcohol, behenyl alcohol, stearic; acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

The preferred oil-in-water emulsions comprise from about 0.05% to about 10%, preferably from about 1% to about 6%, and more preferably from about 1% to about 3% of at least one hydrophilic surfactant which can disperse the hydrophobic materials in the water phase (percentages by weight of the topical carrier). The surfactant, at a minimum, must be hydrophilic enough to disperse in water. Suitable surfactants include any of a wide variety of known cationic, anionic, zwitterionic, and amphoteric surfactants. See, McCutcheon's. Detergents and Emulsifiers, North American Edition (1986), published by Allured Publishing Corporation; U.S. Pat. No. 5,011,681 to Ciotti et al., issued Apr. 30, 1991; U.S. Pat. No. 4,421,769 to Dixon et al. issued Dec. 20, 1983; and U.S. Pat. No. 3,755,560. The exact surfactant chosen depends upon the pH of the composition and the other components present. Preferred are cationic surfactants, especially dialkyl quaternary ammonium compounds, examples of which are described in U.S. Pat. No. 5,151,209 to McCall et al. issued Sep. 29, 1992; U.S. Pat. No. 5,151,210 to Steuri et al. issued Sep. 29, 1992; U.S. Pat. No. 5,120,532; U.S. Pat. No. 4,387,090; U.S. Pat. No. 3,155,591; U.S. Pat. No. 3,929,678; U.S. Pat. No. 3,959,461; McCutcheon's, Detergents & Emulsifiers (North American edition 1979) M.C. Publishing Co.; and Schwartz, et al., Surface Active Agents, Their chemistry and Technology, New York: Interscience Publishers, 1949.

Alternatively, other useful cationic emulsifiers include amino-amides. Nonlimiting examples of these cationic emulsifiers include stearamidopropyl PG-dimonium chloride phosphate, behenamidopropyl PG dimonium chloride, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof.

A wide variety of anionic surfactants are also useful herein. See, e.g., U.S. Pat. No. 3,929,678, to Laughlin et al., issued Dec. 30, 1975. In addition, amphoteric and zwitterionic surfactants are also useful herein.

The preferred oil-in-water emulsion comprises from about 25% to about 98%, preferably from about 65% to about 95%, more preferably from about 70% to about 90% water by weight of the topical carrier.

The hydrophobic phase is dispersed in the continuous aqueous phase. The hydrophobic phase may contain water insoluble or partially soluble materials such as are known in the art, including but not limited to the silicones described herein in reference to silicone-in-water emulsions, and other oils and lipids such as described above in reference to emulsions.

The topical compositions of the subject invention, including but not limited to lotions and creams, may comprise a dermatologically acceptable emollient. Such compositions preferably contain from about 2% to about 50% of the emollient. As used herein, “emollient” refers to a material useful for the prevention or relief of dryness, as well as for the protection of the skin. A wide variety of suitable emollients are known and may be used herein. See, e.g., Sagarin, Cosmetics, Science and Technology, 2nd Edition, Vol. 1, pp. 3243 (1972), which contains numerous examples of materials suitable as an emollient. A preferred emollient is glycerin. Glycerin is preferably used in an amount of from or about 0.001 to or about 20%, more preferably from or about 0.01 to or about 10%, most preferably from or about 0.1 to or about 5%, e.g., 3%.

Lotions and creams according to the present invention generally comprise a solution carrier system and one or more emollients. Lotions typically comprise from about 1% to about 20%, preferably from about 5% to about 10% of emollient; from about 50% to about 90%, preferably from about 60% to about 80% water; and a pharmaceutically effective amount of an agent described herein. A cream typically comprises from about 5% to about 50%, preferably from about 10% to about 20% of emollient; from about 45% to about 85%, preferably from about 50% to about 75% water; and a pharmaceutically effective amount of an agent described herein.

Ointments of the present invention may comprise a simple carrier base of animal or vegetable oils or semi-solid hydrocarbons (oleaginous); absorption ointment bases which absorb water to form emulsions; or water soluble carriers, e.g., a water soluble solution carrier. Ointments may further comprise a thickening agent, such as described in Sagarin, Cosmetics, Science and Technology, 2nd Edition, Vol. 1, pp. 72-73 (1972), incorporated herein by reference, and/or an emollient. For example, an ointment may comprise from about 2% to about 10% of an emollient; from about 0.1% to about 2% of a thickening agent; and a pharmaceutically effective amount of an agent described herein.

By way of non-limiting example, 1000 g of topical cream is prepared from the following types and amounts of ingredients: a pharmaceutically effective amount of an agent disclosed herein, tegacid regular (150 g) (a self-emulsifying glyceryl monostearate from Goldschmidt Chemical Corporation, New York, N.Y.), polysorbate 80 (50 g), spermaceti (100 g), propylene glycol (50 g), methylparaben (1 g), and deionized water in sufficient quantity to reach 1000 gm. The tegacid and spermaceti are melted together at a temperature of 70-80° C. The methylparaben is dissolved in about 500 g of water and the propylene glycol, polysorbate 80, and 6-amino-1,2-dihydro-1-hydroxy-2-imino-4-piperidinopyrimidine free base are added in turn, maintaining a temperature of 75-80° C. The methylparaben mixture is added slowly to the tegacid and spermaceti melt, with constant stirring. The addition is continued for at least 30 minutes with additional stirring until the temperature has dropped to 40-45° C. Finally, sufficient water is added to bring the final weight to 1000 g and the preparation stirred to maintain homogeneity until cooled and congealed.

By way of non-limiting example, 1000 g of a topical ointment is prepared from the following types and amounts of ingredients: a pharmaceutically effective amount of an agent disclosed herein, zinc oxide (50 g), calamine (50 g), liquid petrolatum (heavy) (250 g), wool fat (200 g), and enough white petrolatum to reach 1000 g. Briefly, the white petrolatum and wool fat are melted and 100 g of liquid petrolatum added thereto. The pharmaceutically effective amount of an agent disclosed herein, zinc oxide, and calamine are added to the remaining liquid petrolatum and the mixture milled until the powders are finely divided and uniformly dispersed. The mixture is stirred into the white petrolatum, melted and cooled with stirring until the ointment congeals.

By way of non-limiting example, 1000 g of an ointment, e.g., an ophthalmic ointment, containing a pharmaceutically effective amount of an agent disclosed herein is prepared from the following types and amounts of ingredients: a pharmaceutically effective amount of an agent disclosed herein, light liquid petrolatum (250 g), wool fat (200 g), and enough white petrolatum to reach 1000 g. Briefly, the pharmaceutically effective amount of an agent disclosed herein is finely divided and added to the light liquid petrolatum. The wool fat and white petrolatum are melted together, strained, and the temperature adjusted to 45-50° C. The liquid petrolatum slurry is added, and the ointment stirred until congealed.

By way of non-limiting example, 1000 ml of an aqueous solution containing a pharmaceutically effective amount of an agent disclosed herein is prepared from the following types and amounts of ingredients: a pharmaceutically effective amount of an agent disclosed herein, polyethylene glycol 4000 (120 g) myristyl-gamma-picolinium chloride (0.2 g), polyvinylpyrrolidone (1 g), and enough deionized water to reach 1000 milliliters. Briefly, the ingredients are dissolved in the water and the resulting solution is sterilized by filtration.

By way of non-limiting example, 1000 g of lotion containing a pharmaceutically effective amount of an agent disclosed herein is prepared from the following types and amounts of ingredients: a pharmaceutically effective amount of an agent disclosed herein, N-methyl pyrolidone (40 g), and enough propylene glycol to reach 1000 g.

By way of non-limiting example, an aerosol containing a pharmaceutically effective amount of an agent disclosed herein is prepared from the following types and amounts of materials: a pharmaceutically effective amount of an agent disclosed herein, absolute alcohol (4.37 g), Dichlorodifluoroethane (1.43 g) and dichlorotetrafluoroethane (5.70 g). Briefly, the pharmaceutically effective amount of an agent disclosed herein is dissolved in the absolute alcohol and the resulting solution filtered to remove particles and lint. This solution is chilled to about −30° C. Then, to this is added the chilled mixture of dichlorodifluoromethane and dichlorotetrafluoroethane.

For oral administration, Gelatin capsules or liquid-filled soft gelatin capsules can contain the active ingredient and powdered or liquid carriers, such as lactose, lecithin starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar-coated or film-coated to mask any unpleasant taste and to protect the tablet from the atmosphere, or enteric-coated for selective, targeted disintegration in the gastrointestinal tract. Liquid dosage forms for oral administration can contain coloring and/or flavoring to increase patient acceptance.

In general, sterile water, oil, saline, aqueous dextrose (glucose), polysorbate and related sugar solutions and glycols such as propylene glycol or polyethylene glycols, are suitable carriers for parenteral solutions. Solutions or emulsions for parenteral administration preferably contain about 5-15% polysorbate 80 or lecithin, suitable stabilizing agents and, if necessary, buffer substances. Antioxidizing agents such as, but not limited to, sodium bisulfite, sodium sulfite, or ascorbic acid, either alone or combined, are suitable stabilizing agents. Also useful are citric acid and its salts, and sodium EDTA. In addition, parenteral solutions can contain preservatives including, but not limited to, benzalkonium chloride, methyl- or propyl-paraben, and chlorobutanol.

As will be understood by those in the art, the compositions and pharmaceutical compositions of the invention may be provided in the form of a kit. Kits of the invention comprise one or more specific compositions and/or pharmaceutical compositions of the invention. Optionally, the kit further contains printed instructions as a label or package insert directing the use of such reagents to modify skin pigmentation, i.e., to lighten skin as appropriate to the particular included composition. These compounds are provided in a container designed to prevent contamination, minimize evaporation or drying of the composition, etc. The compounds may or may not be provided in a preset unit dose or usage amount.

The following formulation examples illustrate representative pharmaceutical compositions that may be prepared in accordance with this invention. The present invention, however, is not limited to the following pharmaceutical compositions.

### Formulation 1—Tablets

A compound of the invention may be admixed as a dry powder with a dry gelatin binder in an approximate 1:2 weight ratio. A minor amount of magnesium stearate may be added as a lubricant. The mixture would be formed into 240-270 mg tablets (80-90 mg of active compound per tablet) in a tablet press.

### Formulation 2—Capsules

A compound of the invention may be admixed as a dry powder with a starch diluent in an approximate 1:1 weight ratio. The mixture may be filled into 250 mg capsules (125 mg of active compound per capsule).

### Formulation 3—Liquid

A compound of the invention (125 mg) may be admixed with sucrose (1.75 g) and xanthan gum (4 mg) and the resultant mixture may be blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color could be diluted with water and added with stirring. Sufficient water may then added to produce a total volume of 5 mL.

### Formulation 4—Tablets

A compound of the invention may be admixed as a dry powder with a dry gelatin binder in an approximate 1:2 weight ratio. A minor amount of magnesium stearate may be added as a lubricant. The mixture would be formed into 450-900 mg tablets (150-300 mg of active compound) in a tablet press.

### Formulation 5—Injection

A compound of the invention is dissolved or suspended in a buffered sterile saline injectable aqueous medium to a concentration of approximately 5 mg/mL.

### Formulation 6—Topical

Stearyl alcohol (250 g) and a white petrolatum (250 g) may be melted at about 75° C. and then a mixture of a compound of the invention (50 g) methylparaben (0.25 g), propylparaben (0.15 g), sodium lauryl sulfate (10 g), and propylene glycol (120 g) dissolved in water (about 370 g) could be added and the resulting mixture would be stirred until it congeals.

### General Methods of Preparation

The steroidal compounds of this invention which include certain known drugs or drug like molecules can be purchased from commercial sources and tested for their activities. The steroidal compounds which are not commercially available can be prepared from readily available starting materials using various general methods and procedures known in the art. For example, the compounds may be synthetically prepared from known starting materials by conventional laboratory procedures and protocols. Likewise, those compounds that may be found in existing natural materials may be isolated and/or purified by known procedures, to attain the requisite concentration and content of the active, to be efficacious when formulated into compositions in accordance with the present invention. Such preparations may also be described as formulations or materials that are enriched for the particular compound(s) of the invention, and the present invention embraces such preparations within its scope.

Additionally, as will be apparent to those skilled in the art with respect to the methods of preparation of the compounds of the invention involving organic synthesis, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

### Example 1

### Screening of Compounds in Cultured Murine Melanocytes

The Spectrum Collection library consisting of 2000 drug compounds or natural products was screened to identify novel pigmentation inhibitors or stimulators in cultured murine melanocytes (melan-a). Compounds were dissolved in dimethylsulfoxide (DMSO) to a final concentration of 10 mM. Screening was performed with cultured melanocytes in 24-well plates followed by melanin assay (see below). A minimum change of 50% in melanin formation was established as significant for a pigmentation inhibitor or stimulator. DMSO was used as a negative control and the widely used depigmenting agent, hydroquinone, was used as a positive control on every plate. Primary screening was performed at a final concentration of 1 μM and potential candidates from the primary screening were reconfirmed in duplicate at final concentrations of 1 and 5 μM.

Melan-a cells were plated at 5×10⁴ cells per well in 1 ml of culture media in 24-well plates the day before adding the library compounds. All compounds were added at the indicated final concentrations. Cells were harvested after 72 hours of incubation, and melanin assay was performed.

For further test and mechanism of action studies, the compounds were purchased from either Sigma or Microsource. These compounds were dissolved in dimethylsulfoxide

(DMSO) to a final concentration of 10 mM, and were tested for their effect on melanin synthesis at the indicated final concentrations.

### Example 2

### Melanin Assay

For the primary and secondary screening, cells were harvested and dissolved in 200 μl of 2N NaOH in 20% DMSO at 70° C. A 180-μl aliquot of the resulting solution was measured for absorbance at 490 nm.

For the tested compounds that are involved in the acetylcholine or serotonin pathway or that may demonstrate antimalarial activity, cells are harvested in extraction buffer (1% Triton X-100, 50 mM Tris, 2 mM EDTA, 150 mM NaCl, pH 7.5) containing a complete protease inhibitor cocktail (Roche). The lysates were centrifuged at 14,000 rpm for 10 minutes at 4° C. BCA protein assay kit (Pierce) was used to measure the protein concentrations of the supernatants, and bovine serum albumin was used as a standard. The remaining pellets were incubated with 100 μl ethanol-ether (1:1) for 10 minutes at room temperature. After removing the ethanol-ether, the pellets were dissolved in 200 μl of 2N NaOH in 20% DMSO at 70° C. A 180-μl aliquot of the resulting solution was measured for absorbance at 490 nm. The melanin contents were normalized to the total amount of protein.

The compounds, their structures, inhibition data expressed as % of control remaining, are shown in Table 1, below.

TABLE 1
Activity Data for Compounds Useful as Melanogenesis Modifiers

| | | | | % of | % of |
|---|---|---|---|---|---|
| | | | | control | control |
| | | | | remain- | remain- |
| ID | Name | Structure | MW | ing @ 1 μM | ing @ 5 μM |
| 1 | Conessine | 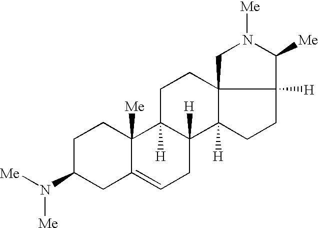 | 356.6 | 73.3 | 32.2 |
| 2 | Con-5-enine, 3β- (dimethylamino)-, (±)- (8Cl) (Registry No: 14546- 12-4) | 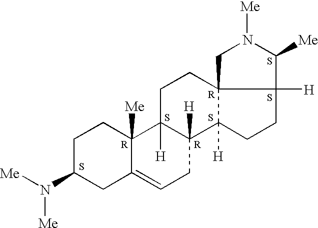 | 356.6 | 78.4 | 38.5 |
| 3 | N,N-Dimethylcon- 5-enin-3-amine | 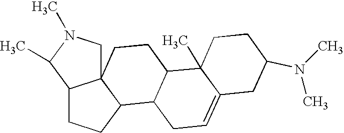 | 356.6 | 59.2 | 28.4 |

### Example 3

### MelanoDerm Pigmentation Assay

The compounds of the invention were tested in the MelanoDerm pigmentation assay, to confirm and demonstrate their activity as inhibitors in a setting that replicates in vivo conditions. MelanoDerm, made by MatTek Corp., is a viable reconstituted three-dimensional human skin equivalent containing normal melanocytes and keratinocytes that are derived from African-American (MEL-B), Asian (MEL-A) or Caucasian (MEL-C) donors. Both MEL-A and MEL-B tissues were used in the current study, and they were maintained in the NMM-113 medium as recommended by the manufacturer.

Conessine (from Sigma) was dissolved in 30% ethanol: 70% propylene glycol to a final concentration of 1.0 mM (equal to 356.6 μg/ml), and this was maintained constant and used on all samples tested. A 25 μl of its aliquot was applied topically to the MelanoDerm tissue (MEL-B) on Days 0, 1, 3, 6, 8 and 10. The MelanoDerm tissues were fed every other day with 5 ml fresh NMM-113. Prior to each application, the tissues were washed with 1 ml PBS to remove any residual test compound. Tissues were fixed on Days 10 and 13 for microscopic analysis and histological evaluation. In addition, duplicate tissues were frozen on Days 10 and 13 for the melanin assay.

Similar experiments were performed on Asian skin equivalent (MEL-A) except: the treatments were applied on Days 0, 1, 3, 6, 8, 10 and 13. Tissues were taken out on Days 13 and 16 for the various assays. 30% ethanol:70% propylene glycol was used as a negative control and the well-known pigmentation inhibitor, arbutin (at concentration of 3 mg/ml), was used as a positive control.

The experiments were repeated twice on both MEL-A and MEL-B tissues from different lots to make sure that the results are reproducible (study 1 or 2). For each experiment, six tissues were treated with conessine, and six were treated with vesicle (30% ethanol:70% PEG) or arbutin if applicable. For MEL-B, on day 10, three tissues under each treatment condition were taken out, and one was used for histological studies and the other two were used for the melanin assay. The same protocol was followed with the MEL-B samples after 13 days' treatment and MEL-A after either 13 or 16 days' treatments.

For the Histological Studies:

Procedure 1: the effects of conessine on melanin synthesis in MEL-A or B were evaluated by light microscopy (views from the top surface of the tissue).
Procedure 2: the distribution of melanin in the treated-MEL-A or B was accessed by image analysis using Fontana-Masson stained histological sections (views from the side of the tissue).

For the melanin tissues, the melanin content of each individual tissue was determined, and the final data show the average melanin content of 2 tissues treated under identical conditions.

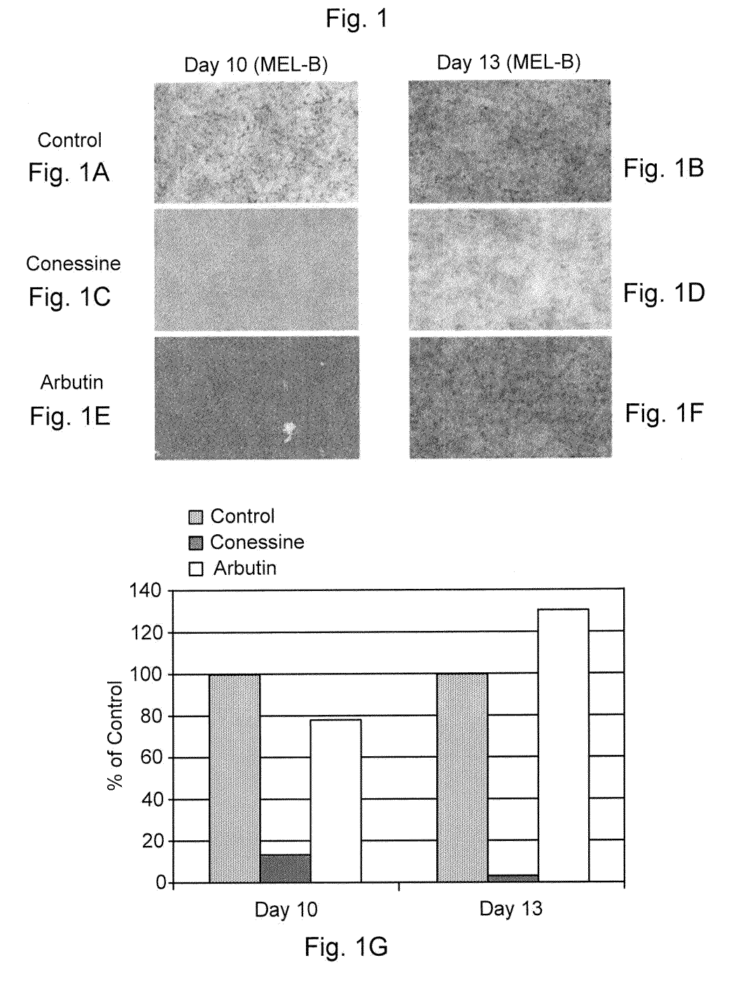

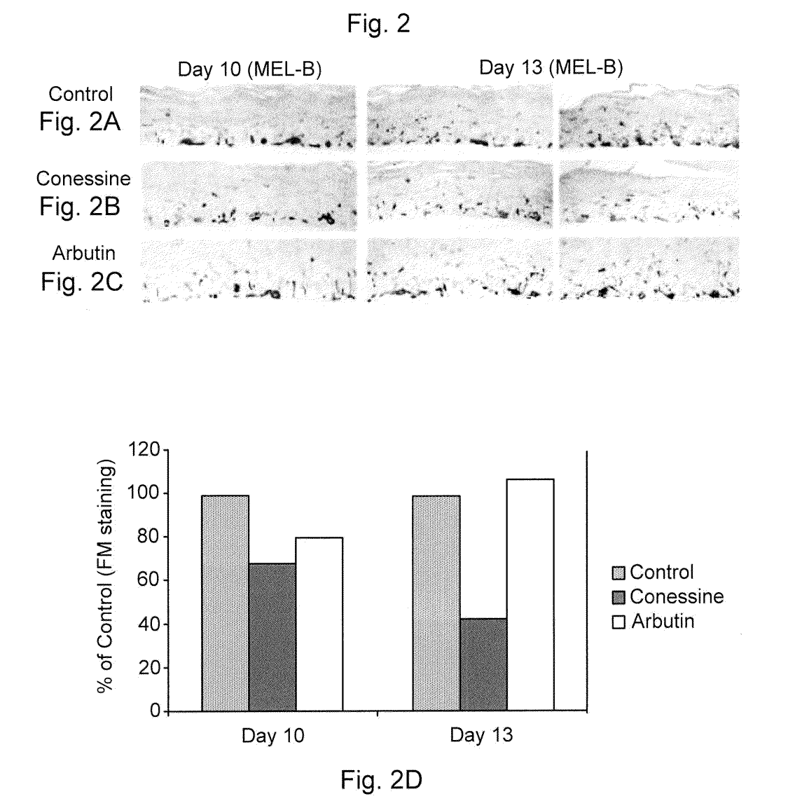

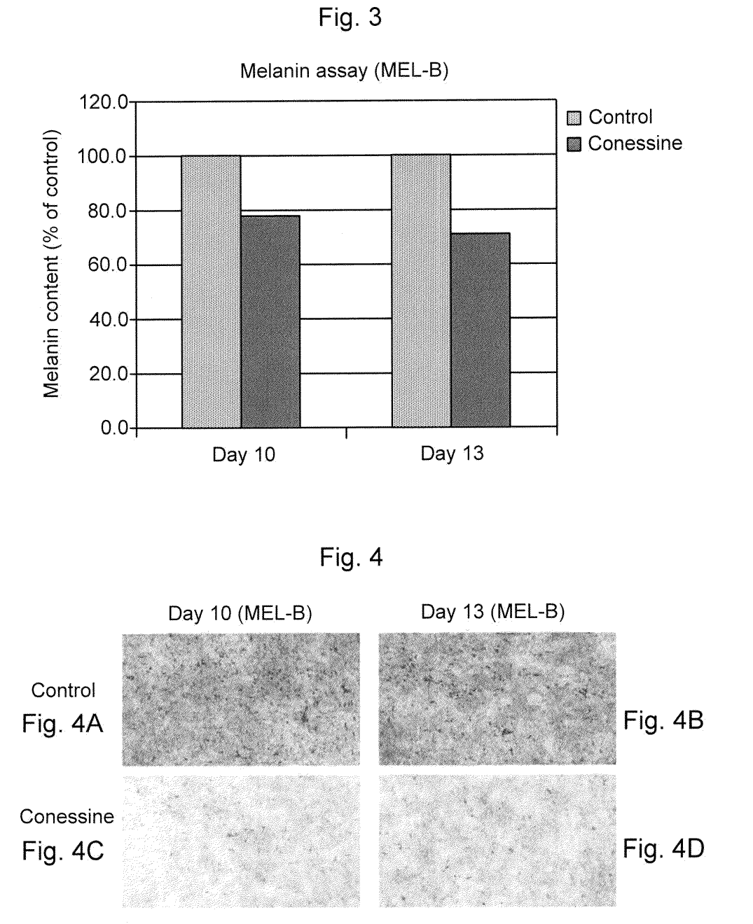

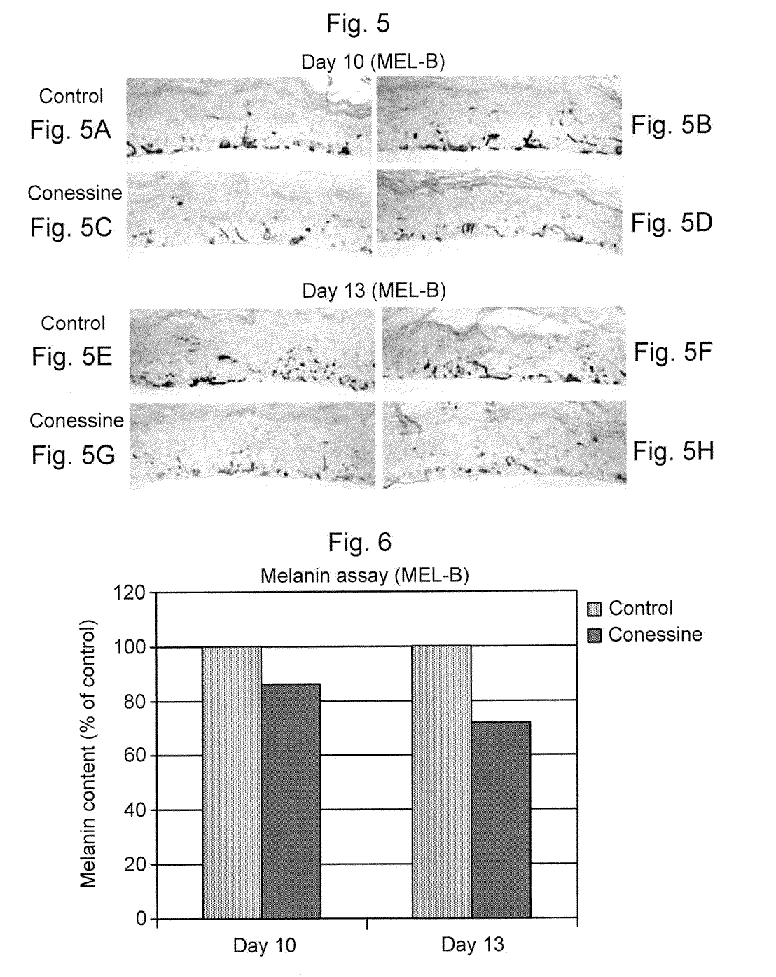

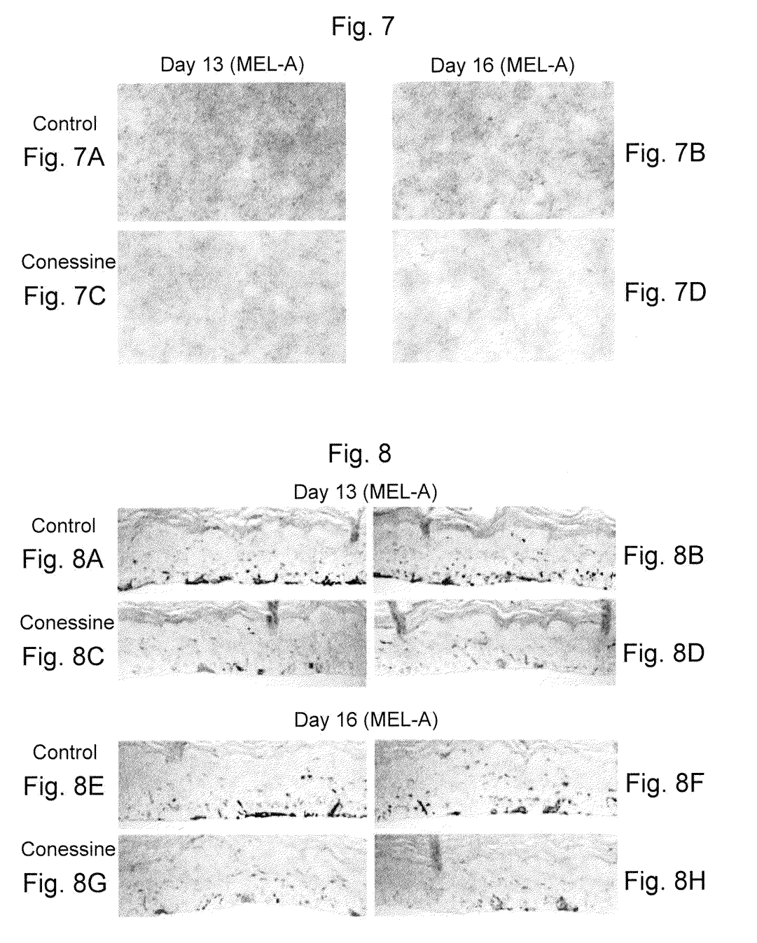

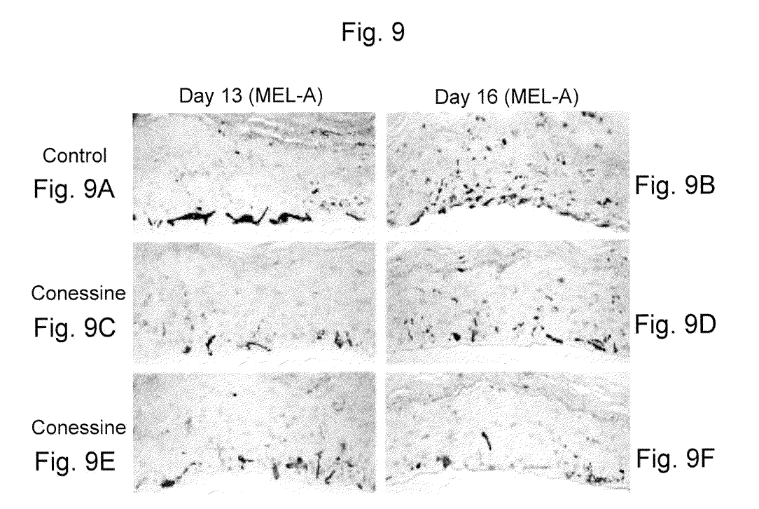

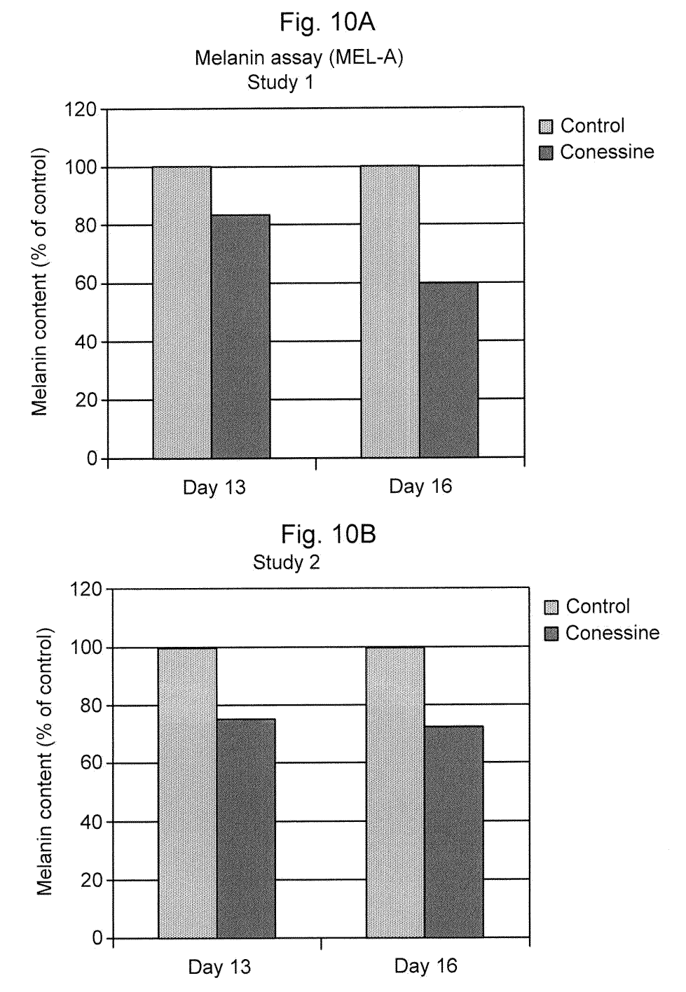

The results of the assays of both skin equivalents are set forth in FIGS. 1A-10B, and clearly demonstrate that the compounds of the invention achieve skin lightening in all of the samples tested.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

From the foregoing description, various modifications and changes in the compositions and methods of this invention will occur to those skilled in the art. All such modifications coming within the scope of the appended claims are intended to be included therein.

All publications, including but not limited to patents and patent applications, cited in this specification are herein incorporated by reference as if each individual publication were specifically and individually indicated to be incorporated by reference herein as though fully set forth.

The chemical names of compounds given in this application were generated using various commercially available chemical naming software tools including MDL's ISIS Draw Autonom Software tool, and were not verified. Particularly, in the event of inconsistency, the depicted structure governs.

What is claimed is:

1. A method for inhibiting melanogenesis by melanocytes comprising administering to the melanocytes an effective amount of a compound of formula I 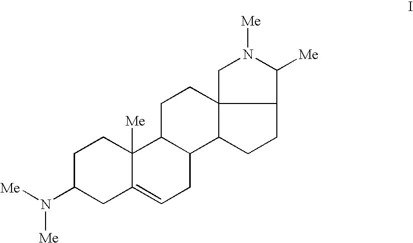
or a pharmaceutically acceptable salt, solvate or prodrug thereof, and stereoisomers, tautomers and isotopic variants thereof.

2. The method of claim 1 wherein the compound is conessine.

3. The method of any one of the preceding claims, wherein the melanocytes are mammalian melanocytes.

4. A compound for use as an inhibitor of melanogenesis comprising a compound of either of claim 1 or 2.

5. A composition comprising a pharmaceutically acceptable carrier and a pharmaceutically effective amount of a compound of either of claim 1 or 2.

6. The composition of claim 5 wherein the carrier is a parenteral carrier, oral or topical carrier.

7. A topical formulation comprising a composition for cosmetic or dermatological use, said composition comprising a compound of either of claim 1 or 2, and an acceptable carrier.

8. A method for preventing, treating, ameliorating or managing a disease or condition involving undesired or aberrant melanogenesis, which comprises administering to a patient in need or desirous of such prevention, treatment, amelioration or management, a prophylactically or therapeutically effective melanogenesis-inhibiting amount of a compound of claims for 2, or a composition of any of claims 3-7.

9. The method of claim 8, wherein the compound or composition is administered to lighten or reduce pigmentation levels.

10. The method of claim 8, wherein the compound or composition is administered to lighten or reduce pigmentation levels of hyperpigmented sites on skin.

11. A method for altering or restoring pigmentation in mammalian skin, hair, wool or fur comprising administering to the mammalian skin, hair, wool or fur an amount of a composition comprising:
a) an amount, which is effective to alter or restore pigmentation in mammalian skin, hair, wool or fur, of one or more compounds of either of claim 1 or 2, or a composition of claims 5-7; and b) a suitable carrier, wherein the amount is effective to alter or restore pigmentation in mammalian skin, hair, wool or fur.

12. The method of claim 10, wherein the compound is a melanogenesis inhibitor.

13. The method of claim 11, wherein the melanogenesis inhibitor is administered to lighten or reduce pigmentation levels of the mammalian skin, hair, wool or fur.

14. A compound of either of claim 1 or 2, or a pharmaceutically acceptable salt or solvate thereof for use as a pharmaceutical or a medicament.

15. The use of a compound or a pharmaceutically acceptable salt, solvate or composition thereof, as defined in either of claim 1 or 2, for the manufacture of a medicament to treat a disease or condition for which a melanogenesis inhibitor is indicated.

16. A use according to claim 14 where the disease is selected from hyperpigmentation or uneven pigmentation disorders such as age spots, freckles, drug-induced hyperpigmentation, post-inflammatory hyperpigmentation as seen in acne, seborrheic keratoses, melasma and chloasma.

17. A method of treatment of a mammal, including a human being, to treat a disease for which a melanogenesis inhibitor is indicated, including treating said mammal with an effective amount of a compound or with a pharmaceutically acceptable salt, solvate or composition thereof, as defined in any of claims 1-4.

18. A combination of a compound as defined in either of claim 1 or 2, and a like-acting agent.

19. The combination of claim 18, wherein said like acting agent is selected from a cosmetic ingredient and a pharmacologically active agent.

20. The combination of claim 19, wherein the cosmetic ingredient is a skin lightener.

21. The combination of claim 20, wherein said pharmacologically active agent is selected from another melanogenesis inhibitor.

22. A pharmaceutical composition useful to treat a disease for which a melanogenesis inhibitor is indicated, comprising a therapeutically effective amount of a combination of any one of claims 18-21, wherein the like acting agent is a pharmacologically active agent.

23. The pharmaceutical composition of claim 22, wherein the like-acting agent is a skin lightening compound.

24. A topical formulation comprising a composition for cosmetic or dermatological use, the said composition comprising a cosmetically and/or dermatologically effective amount of a combination of any one of claims 18-21, wherein the like acting agent is a cosmetically active agent.

25. The topical formulation of claim 24, wherein the like-acting agent is a skin lightening compound.

26. A method for preventing, treating, ameliorating or managing a disease or condition involving undesired or aberrant melanogenesis, which comprises administering to a patient in need or desirous of such prevention, treatment, amelioration or management, a pharmaceutical composition comprising prophylactically or therapeutically effective melanogenesis-inhibiting amount of a combination of any one of claims 18-21, wherein the like acting agent is a pharmacologically active agent.

27. The method of claim 26, wherein the like-acting agent is a skin lightening compound.

28. A method for altering or restoring pigmentation in mammalian skin, hair, wool or fur comprising administering to the mammalian skin, hair, wool or fur an amount of a composition comprising a pigment restoring or altering-effective amount of a combination of any one of claims 18-21, wherein the like acting agent is a cosmetically active agent.

29. The method of claim 28, wherein the like-acting agent is a skin lightening compound.
